(19) United States
(12) Patent Application Publication
(10) Pub. No.: US 2025/0285438 A1
(43) Pub. Date: Sep. 11, 2025

(54) Metrics and Event Detection Using Multi-Modal Data

(71) Applicant: AURIS HEALTH, INC., Santa Clara, CA (US)

(72) Inventors: Duke LIN, Redwood City, CA (US); Elif AYVALI, Redwood City, CA (US); Menglong YE, Mountain View, CA (US)

(21) Appl. No.: 18/599,180

(22) Filed: Mar. 7, 2024

(51) Int. Cl. G06V 20/40; A61B 34/20; A61B 34/30; G06V 10/70; G06V 10/764; G06V 20/52; G06V 20/70; G16H 30/20; G16H 40/67

(52) U.S. Cl. CPC G06V 20/41; A61B 34/20; G06V 10/764; G06V 10/768; G06V 20/44; G06V 20/46; G06V 20/52; G06V 20/70; G16H 30/20; G16H 40/67; A61B 2034/2057; A61B 2034/2065; A61B 2034/303; G06V 2201/034

(57) ABSTRACT

A system for extracting information of objects from video captured during a medical procedure that includes an image repository configured to store image data representing views within a luminal network, a log repository configured to store commands and/or states associated with an object within the luminal network, and control circuitry. The control circuitry can be configured to generate change data representing changes of visual states of the object over a time period, access the log repository to determine logs including at least one command or at least one state associated with the object over the time period, and generate contextual information associated with the object based at least in part on (i) the change data and (ii) the at least one command or the at least one state associated with the object.

### BACKGROUND

### Field

The present disclosure relates to the field of medical procedures.

### Description of Related Art

Various medical procedures involve the use of one or more scope and/or percutaneous access instruments. The scope or the instruments can capture and provide image data. Robotics systems controlling the scope or the instruments can provide log data relating to robotic commands or robotic states.

### SUMMARY

Described herein are systems, devices, and methods to generate contextual information from various medical procedures performed using a robotic medical system. Multi-modal data, including robot data, image data, and sensor data, may be made accessible by the robotic medical system in connection with a medical procedure, such as bronchoscopy, nephroscopy, or other procedure accessing an anatomical feature, for example. Context may be gained by examining data of one mode in combination with data of another mode. For example, vision data may be matched with robot data to generate contextual information, representing the context, that was otherwise not available when examining only vision data or the robot data. Various functionalities of the robotic medical system may be controlled (e.g., enabled, disabled, throttled, etc.) based on the contextual information. Accordingly, contextual information generation in accordance with aspects of the present disclosure can provide valuable insights regarding medical procedures and assist performance of the medical procedures.

In some aspects, the techniques described herein relate to a system for extracting information of objects from video captured during a medical procedure, the system including: an image repository configured to store image data representing views within a luminal network, the views captured with an imaging device; a log repository configured to store commands and/or states associated with an object within the luminal network; and control circuitry configured to: generate change data representing changes of visual states of the object over a time period based at least in part on the image data; access the log repository to determine logs including at least one command or at least one state associated with the object over the time period; and generate contextual information associated with the object based at least in part on (i) the change data and (ii) the at least one command or the at least one state associated with the object.

In some aspects, the techniques described herein relate to a system, wherein the control circuitry is further configured to: assign, using a machine learning classifier, a semantic label to the object in one or more image frames of the image data.

In some aspects, the techniques described herein relate to a system, wherein the control circuitry is further configured to: determine the object is a LASER, a basket, a Percutaneous Antegrade Urethral Catheter (PAUC), a ureteral access sheath (UAS), a needle, an anatomical feature, or a stone.

In some aspects, the techniques described herein relate to a system, wherein the control circuitry is further configured to: determine a medical procedure or a phase of the medical procedure.

In some aspects, the techniques described herein relate to a system, wherein the control circuitry is further configured to: based on the change data, determine a starting image frame and an ending image frame from one or more image frames of the image data, the change data including at least one of: (i) visibility of the object, (ii) movement of the object, or (iii) a detected size, shape, or count of the object.

In some aspects, the techniques described herein relate to a system, wherein the accessing the log repository further includes: filter the log repository to select the logs including the at least one command or the at least one state associated with the object; determine a timestamp from the selected logs; and determine a starting image frame for one or more image frames of the image data based on the timestamp.

In some aspects, the techniques described herein relate to a system, wherein the control circuitry is further configured to: select an image frame associated with a timestamp from one or more image frames of the image data; and index the image frame with the determined contextual information, the contextual information including at least one of: (i) a medical procedure, (ii) a phase of the medical procedure, (iii) a result of the medical procedure, (iv) a result of the phase of the medical procedure, (v) a visual state of the image data, (vi) visibility of the object, or (vii) a relative position of the object in relation to another object.

In some aspects, the techniques described herein relate to a system, the control circuitry is further configured to: receive a selection query including the contextual information; and in response to the receiving of the selection query, provide the timestamp or the image frame.

In some aspects, the techniques described herein relate to a system, wherein the commands include at least one of: insertion, retraction, LASER activation, articulation, basket open or closure, aspiration, irrigation, or puncture.

In some aspects, the techniques described herein relate to a system, wherein the states include at least one of: kinematics, position, orientation, usage time, number of activations, protrusion length, number of stone retrievals, treatment time, articulation duration, blind driving, backflow, LASER fires or LASER misfires, or successful puncture.

In some aspects, the techniques described herein relate to a system, wherein the control circuitry is further configured to: based on the determined contextual information, enable an operational functionality of the object.

In some aspects, the techniques described herein relate to a system, wherein the control circuitry is further configured to: cause a display to present a warning based on the determined contextual information.

In some aspects, the techniques described herein relate to a system, wherein the log repository is configured to further store electromagnetic (EM) sensor data and the contextual information is generated based at least in part on the EM sensor data.

In some aspects, the techniques described herein relate to a system, wherein the control circuitry is further configured to: access a voice recording captured by a recording device; converting the voice recording into text; and indexing a first segment of the image data with a first segment of the text, the first segment associated with a timestamp.

In some aspects, the techniques described herein relate to a system, the control circuitry is further configured to: receive a selection query including the first segment of the text; and in response to the receiving of the selection query, provide the timestamp or the first segment of the image data.

In some aspects, the techniques described herein relate to a method for extracting information of objects from image captured during a medical procedure, the method including: accessing image data representing a view within a luminal network, the image data accessed from an image repository configured to store the image data; accessing commands and/or states associated with a medical tool configured to operate within the luminal network from a log repository; generating change data representing changes of visual states of an object over a time period based at least in part on the image data; determining logs including at least one command or at least one state associated with the medical tool over the time period; and generating contextual information associated with the object based at least in part on (i) the change data and (ii) the at least one command or the at least one state associated with the medical tool.

In some aspects, the techniques described herein relate to a method, further including: filtering the log repository to select the logs including the at least one command or the at least one state associated with the medical tool; determining a timestamp from the selected logs; and determining a starting image frame for one or more image frames of the image data based on the timestamp.

In some aspects, the techniques described herein relate to a method, further including: selecting an image frame associated with a timestamp from one or more image frames of the image data; and indexing the image frame with the determined contextual information, the contextual information including at least one of: (i) the at least one command or (ii) the at least one state, the at least one command or the at least one state associated with the medical tool.

In some aspects, the techniques described herein relate to a method, further including: receiving a selection query including the contextual information; and in response to the receiving of the selection query, providing the timestamp or the image frame.

In some aspects, the techniques described herein relate to a system for determining metrics and events of objects from image captured during a medical procedure, the system including: control circuitry communicatively coupled to (i) an image repository configured to store image data representing views within a luminal network, the views captured with an imaging device, and (ii) a log repository configured to store data from sensors other than the imaging device, the control circuitry configured to: generate change data representing changes of visual states of an object over a time period based at least in part on the image data; access the log repository to determine logs including sensor data associated with the object over the time period; and determine metrics and events associated with the object based at least in part on (i) the change data and (ii) the sensor data associated with the object.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example a robotic medical system in accordance with one or more embodiments.

FIG. 2 illustrates an example table-based robotic medical system in accordance with one or more embodiments.

FIG. 3 illustrates an example control system in accordance with one or more embodiments.

FIG. 4 illustrates an example robotic system in accordance with one or more embodiments.

FIG. 5 illustrates an example robotic instrument feeder in accordance with one or more embodiments.

FIG. 6 illustrates an example robotically-controllable endoscope in accordance with one or more embodiments.

FIG. 7 illustrates an example block diagram of a multi-modal contextual information generator pipeline in accordance with one or more embodiments.

FIG. 8 illustrates an example system including a context management framework in accordance with one or more embodiments.

FIG. 9 illustrates example classifications of various medical tools in accordance with one or more embodiments.

FIG. 10 illustrates hard and soft masks usable in labelling of various objects in accordance with one or more embodiments.

FIG. 11 illustrates an image-based object segmentation framework in accordance with one or more embodiments.

FIGS. 12A, 12B, and 12C illustrate student-teacher training paradigm in accordance with one or more embodiments.

FIGS. 13A and 13B illustrate example metrics and events in accordance with one or more embodiments.

FIG. 14 illustrates an example timeline of multi-modal data and generated contextual information in accordance with one or more embodiments.

FIG. 15 illustrates a flow diagram illustrating a process for generating contextual information of an object in accordance with one or more embodiments.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts features associated with separate figures that are identified by common reference numbers are not related and/or similar with respect to at least certain aspects.

### Overview

The present disclosure relates to systems, devices, and methods for generating contextual information relating to medical procedures from multi-modal data and applications of the contextual information during and after the medical procedures. Although certain aspects of the present disclosure are described in detail herein in the context of renal, urological, and/or nephrological procedures, such as kidney stone removal/treatment procedures, it should be understood that such context is provided for convenience and clarity, and contextual information generation and application concepts disclosed herein are applicable to any suitable medical procedures.

Multi-modal data can refer to datasets that involve information from multiple modes or sources. Each mode represents a different way of sensing, capturing, or representing data. Example modes can include text data scanned with optical character recognition (OCR), image data captured with cameras, sensor data collected as sensor readings, audio or video data recorded, and more. Regarding a robotic medical system, multi-modal data can include first data (e.g., image data, robot data, sensor data, etc.) selected from a first data source and second data selected from a second data source distinct from the first data source. The multi-modal data may be accessed in real-time or accessed from logs or repositories.

Image data may include endoview images captured by a camera positioned at or near a distal end of an endoscope (e.g., vision data) or diagnostic images (e.g., X-ray, CT scans, MRI, or the like). Robot data may include any command instructed to any component of the robotic medical system, such as one or more robotic arms, end effectors, actuators, medical tools, etc. Robot data may also include any robotic states of any component of the robotic medical system, such as kinematic states of joints or activation states (e.g., open/close state of a medical tool). Sensor data may include any measurements of physical properties (e.g., temperature, pressure/force, proximity, light, motion, sound, humidity, electromagnetic (EM) field variations, etc.) provided by one or more sensors of the robotic medical system. As an example of a sensor and sensor data, an EM sensor (or tracker) comprising of one or more sensor coils embedded in one or more locations and orientations in an endoscope can measure the variation in the EM field created by one or more EM field generators. The magnetic field induces small currents in the sensor coils of the EM sensor, which may be analyzed to determine the distance and angle between the EM sensor and the EM field generator.

The multi-modal data can provide contextual information that is otherwise unavailable from a single mode of data. For example, when operating a basket tool at a distal tip of a scope, logged robot data of open/close commands may indicate how many grasp attempts were made and vision data may confirm whether any of grasp attempts captured stone(s). Such contextual information, referred herein as “metrics” and “events”, are not readily determinable with only the vision data or the robot data.

The metrics and events may serve to help improve control and safety of the robotic medical system. For instance, when robotic data indicate driving of a medical tool without proper visibility from vision data, thereby indicating a “blind driving” event, a warning may be generated and insertion may be slowed or stopped. Additionally, when robot data/sensor data indicate safe positioning of a medical tool within an access sheath with vision data showing no visibility of the medical tool, the medical tool may be retracted with increased speed (e.g., turn on “fast retraction”) to significantly speed up the medical procedure.

In addition to improved insights, functionality, and safety, the metrics and events can be used to segment, categorize, and index a portion of the multi-modal data. For example, vision data can be used to time-window a first time period reflecting navigation toward (e.g., increasing insertion length) a stone and, once the stone is visually identified, a second time period reflecting treatment. Similarly, an end of treatment may be “clocked” when retraction starts with no stone left visible. Video having one or more image frames of the vision data can be segmented into phases or workflow steps and segmented accordingly. As another example, one or more image frames can be categorized or indexed with successful or failed grasp attempts. Once indexed, a physician may readily review a completed medical procedure by querying/filtering based on the categorization/indexing to identify a particular segment of the video. Accordingly, the contextual information determination using multi-modal data can provide various advantages over existing systems.

### Medical System

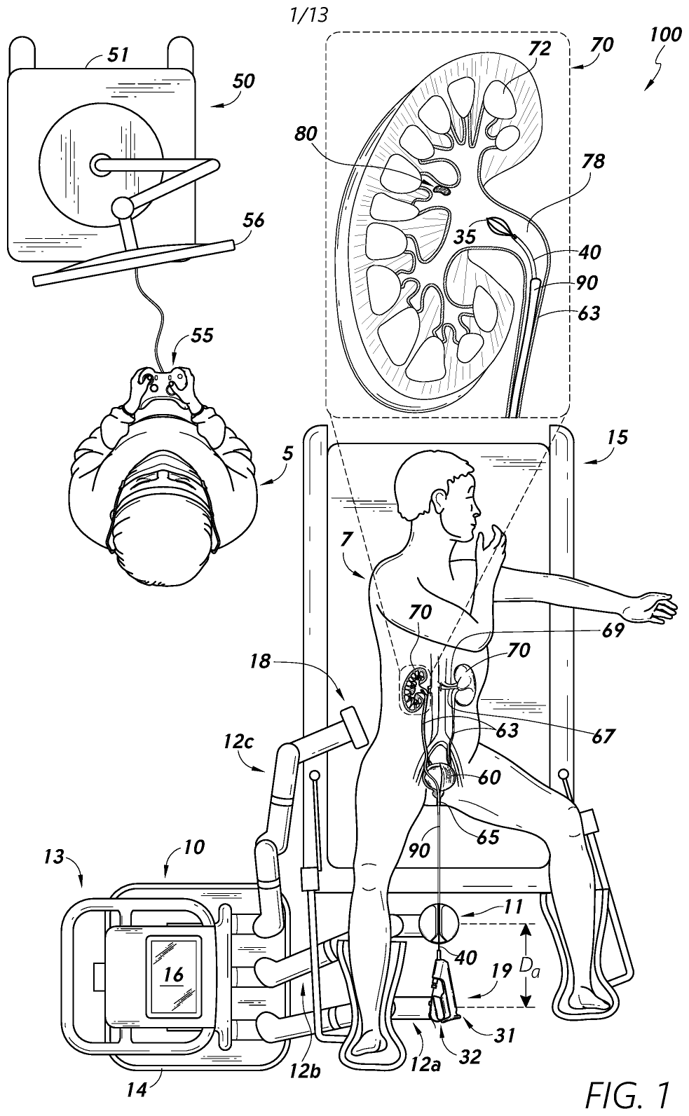

FIG. 1 illustrates an example robotic medical system 100 for performing various medical procedures in accordance with aspects of the present disclosure. The robotic medical system 100 may be used for, for example, endoscopic (e.g., ureteroscopic) procedures. As referenced and described above, certain ureteroscopic procedures involve the treatment/removal of kidney stones. In some implementations, kidney stone treatment can benefit from the assistance of certain robotic technologies/devices. Robotic medical solutions can provide relatively higher precision, superior control, and/or superior hand-eye coordination with respect to certain instruments compared to strictly-manual procedures. For example, robotic-assisted ureteroscopic access to the kidney in accordance with some procedures can advantageously enable a urologist to articulate a ureteroscope using robotically-controlled gears/drives coupled to a handle/base portion of the ureteroscope. Although the system 100 of FIG. 1 is presented in the context of a ureteroscopic procedure, it should be understood that the principles disclosed herein may be implemented in any type of endoscopic procedure.

The robotic medical system 100 includes a robotic system 10 (e.g., mobile robotic cart) configured to engage with and/or control a medical instrument 19 (e.g., endoscope/ureteroscope) including a proximal handle/base 31 and a shaft 40 coupled to the handle 31 at a proximal portion thereof to perform a direct-entry procedure on a patient 7. The term “direct-entry” is used herein according to its broad and ordinary meaning and may refer to any entry of instrumentation through a natural or artificial opening in a patient's body. For example, with reference to FIG. 1, the direct entry of the scope/shaft 40 into the urinary tract of the patient 7 may be made through the urethra 65. The term “patient” is used herein to refer to live patient as well as any subjects to which the present disclosure may be applicable. For example, the “patient” may refer to subjects including physical anatomic models (e.g., anatomical education model, anatomical model, medical education anatomy model, etc.) used in dry runs, models in computer simulations, or the like that covers non-live patients or subjects.

It should be understood that the direct-entry instrument 19 may be any type of shaft-based medical instrument, including an endoscope (such as a ureteroscope), catheter (such as a steerable or non-steerable catheter), nephroscope, laparoscope, or other type of medical instrument. Embodiments of the present disclosure relating to ureteroscopic procedures for removal of kidney stones through a ureteral access sheath (e.g., the ureteral access sheath 90) are also applicable to solutions for removal of objects through percutaneous access, such as through a percutaneous access sheath. For example, instrument(s) may access the kidney percutaneously through a percutaneous access sheath to capture and remove kidney stones. The term “percutaneous access” is used herein according to its broad and ordinary meaning and may refer to entry, such as by puncture and/or minor incision, of instrumentation through the skin of a patient and any other body layers necessary to reach a target anatomical location associated with a procedure (e.g., the calyx network of the kidney 70).

The robotic medical system 100 includes a control system 50 configured to interface with the robotic system 10, provide information regarding the procedure, and/or perform a variety of other operations. For example, the control system 50 can include one or more display(s) 56 configured to present certain information to assist the physician 5 and/or other technician(s) or individual(s). The robotic medical system 100 can include a table 15 configured to hold the patient 7. The system 100 may further include an electromagnetic (EM) field generator 18, which may be held by one or more of the robotic arms 12 of the robotic system 10 or may be a stand-alone device mounted to the table 15. Although the various robotic arms 12 are shown in various positions and coupled to various tools/devices, it should be understood that such configurations are shown for convenience and illustration purposes, and such robotic arms may have different configurations over time and/or at different points during a medical procedure. Furthermore, the robotic arms 12 may be coupled to different devices/instruments than shown in FIG. 1.

Articulation of the shaft 40 may be controlled robotically, such as through operation of an end effector associated with the robot arm 12a, wherein such operation may be controlled by the control system 50 and/or robotic system 10. The term “end effector” is used herein according to its broad and ordinary meaning and may refer to any type of robotic manipulator device, component, and/or assembly. In implementations in which an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. In some contexts, the combination of a robotic end effector and adapter may be referred to as an instrument manipulator assembly 150, wherein such assembly may or may not also include a medical instrument (or instrument handle/base) physically coupled to the adapter and/or end effector. The terms “robotic manipulator” and “robotic manipulator assembly” are used according to their broad and ordinary meanings, and may refer to a robotic end effector and/or sterile adapter or other adapter component coupled to the end effector, either collectively or individually. For example, the terms “robotic manipulator” and “robotic manipulator assembly” may refer to an instrument device manipulator (IDM) including one or more drive outputs, whether embodied in a robotic end effector, sterile adapter, and/or other component(s). The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

In an example use case, if the patient 7 has a kidney stone (or stone fragment) 80 located in a kidney 70, the physician 5 may perform a procedure to remove the stone 80 through the urinary tract (63, 60, 65). In some embodiments, the physician 5 can interact with the control system 50 and/or the robotic system 10 to cause/control the robotic system 10 to advance and navigate the medical instrument shaft 40 (e.g., a scope) from the urethra 65, through the bladder 60, up the ureter 63, and into the renal pelvis 78 and/or calyx network of the kidney 70 where the stone 80 is located. The control system 50 can provide information via the display(s) 56 that is associated with the medical instrument 40, such as real-time endoscopic images captured therewith, and/or other instruments of the system 100, to assist the physician 5 in navigating/controlling such instrumentation.

With further reference to the robotic medical system 100, the medical instrument shaft 40 (e.g., scope, directly-entry instrument, etc.) can be advanced into the kidney 70 through the urinary tract. Specifically, a ureteral access sheath 90 may be disposed within the urinary tract to an area near the kidney 70. The shaft 40 may be passed through the ureteral access sheath 90 to gain access to the internal anatomy of the kidney 70, as shown. The distal portion of the scope/shaft 40 deployed from the sheath 90 may be articulatable to allow the surgeon 5 to use inputs of the control device 55 to cause the robotic system 10 to articulate the shaft 40 towards the target kidney stone. Once at the site of the kidney stone 80 (e.g., within a target calyx 75 of the kidney 70 through which the stone 80 is accessible), the medical instrument 19 and/or shaft 40 thereof can be used to channel/direct the basketing device 30 to the target location. Once the stone 80 has been captured in the distal basket portion 35 of the basketing device/assembly 30, the utilized ureteral access path may be used to extract the kidney stone 80 from the patient 7. Advancement and retraction of the scope shaft 40 can be implemented by an instrument feeder device 11, which may be coupled to an end effector actuator, as shown.

The various scope/shaft-type instruments disclosed herein, such as the shaft 40 of the system 100, can be configured to navigate within the human anatomy, such as within a natural orifice or lumen of the human anatomy. The terms “scope” and “endoscope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise an at least partially rigid and/or flexible tube, and may be dimensioned to be passed within an outer sheath, catheter, introducer, or other lumen-type device, or may be used without such devices.

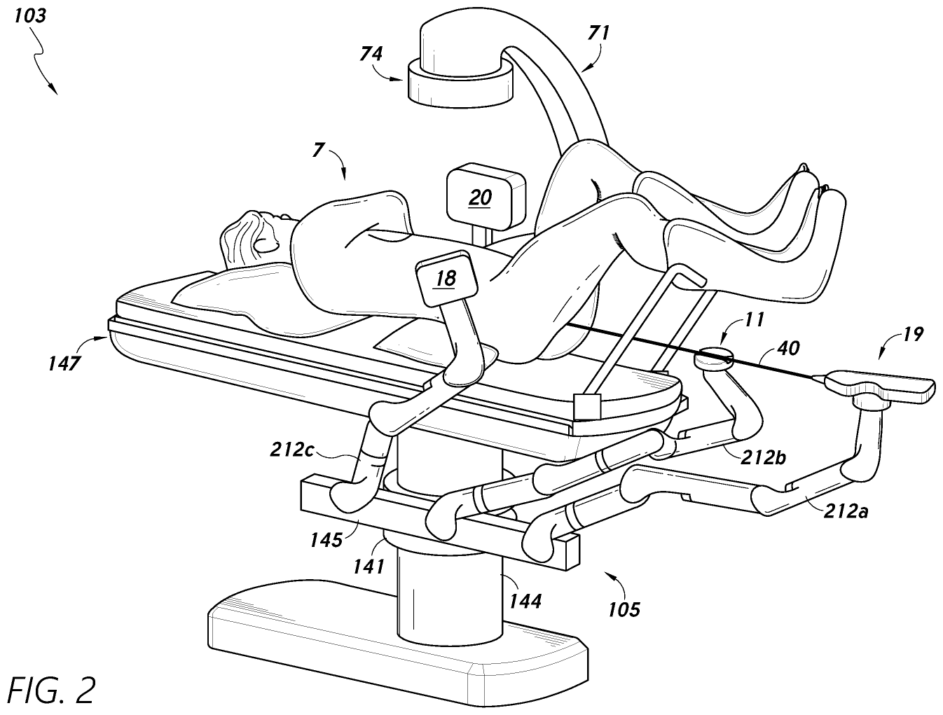

FIG. 2 illustrates a table-based robotic system 103 in accordance with one or more embodiments of the present disclosure. The system 103 incorporates robotic components 105 with a table/platform 147, thereby allowing for a reduced amount of capital equipment within the operating room compared to some cart-based robotic systems, which can allow greater access to the patient 7 in some instances. Much like in cart-based systems, the instrument device manipulator assemblies associated with the robotic arms 212 of the system 103 may generally comprise instruments and/or instrument feeders that are designed to manipulate an elongated medical instrument/shaft, such as an endoscope 40 or the like, along a virtual rail/path.

As shown, the robotic-enabled table system 103 can include a column 144 coupled to one or more carriages 141 (e.g., ring-shaped movable structures), from which the one or more robotic arms 212 may emanate. The carriage(s) 141 may translate along a vertical column interface that runs at least a portion of the length of the column 144 to provide different vantage points from which the robotic arms 212 may be positioned to reach the patient 7. The carriage(s) 141 may rotate around the column 144 in some embodiments using a mechanical motor positioned within the column 144 to allow the robotic arms 212 to have access to multiples sides of the table/platform 147. Rotation and/or translation of the carriage(s) 141 can allow the system 103 to align the medical instruments, such as endoscopes 40 and sheaths, into different access points on the patient 7. By providing vertical adjustment, the robotic arms 212 can advantageously be configured to be stowed compactly beneath the table/platform 147 of the table system 103 and subsequently raised during a procedure.

The robotic arms 212 may be mounted on the carriage(s) 141 through one or more arm mounts 145, which may comprise a series of joints that may individually rotate and/or telescopically extend to provide additional configurability to the robotic arms 212. The column 144 structurally provides support for the table/platform 147 and a path for vertical translation of the carriage(s) 141. The column 144 may also convey power and control signals to the carriage(s) 141 and/or the robotic arms 212 mounted thereon. The system 103 can include certain control circuitry configured to control driving and/or articulation of the instrument shaft 40 using an end effector of one of the robotic arms 212. The robotic-enabled table system 103 may include the robotically-held EM field generator 18 or a table-mounted EM field generator 20. In some embodiments, the table-mounted EM field generator made positioned over or under the surface of the table 15. Although a control tower/system is not shown in FIG. 2 for visual clarity, it should be understood that the system 103 may have a control tower/system as in any embodiment disclosed herein.

Various positioning/imaging modalities may be implemented to provide images/representations of the anatomical space. Suitable imaging subsystems include, for example, X-ray, fluoroscopy, CT, PET, PET-CT, CT angiography, Cone-Beam CT, 3DRA, single-photon emission computed tomography (SPECT), MRI, Optical Coherence Tomography (OCT), and ultrasound. One or both of pre-procedural and intra-procedural images may be acquired. In some embodiments, the pre-procedural and/or intra-procedural images are acquired using a C-arm fluoroscope. In connection with some embodiments, particular positioning and imaging systems/modalities are described; it should be understood that such description may relate to any type of positioning system/modality.

The system 100 is illustrated as including a fluoroscopy system, which includes an X-ray generator 75 and an image detector 74 (referred to as an “image intensifier” in some contexts; either component 74, 75 may be referred to as a “source” or “emitter” herein), which may both be mounted on a moveable/rotatable structure, such as the C-arm 71. In some instances, the fluoroscopy system and any portions thereof may be referred as an imaging device. The control system 50 or other system/device may be used to store and/or manipulate images generated using the fluoroscopy system. In some embodiments, the bed 15 is radiolucent, such that radiation from the generator 75 may pass through the bed 15 and the target area of the patient's anatomy, wherein the patient 7 is positioned between the ends of the C-arm 71. The fluoroscopy system 70 may be implemented to allow live images to be viewed to facilitate image-guided surgery.

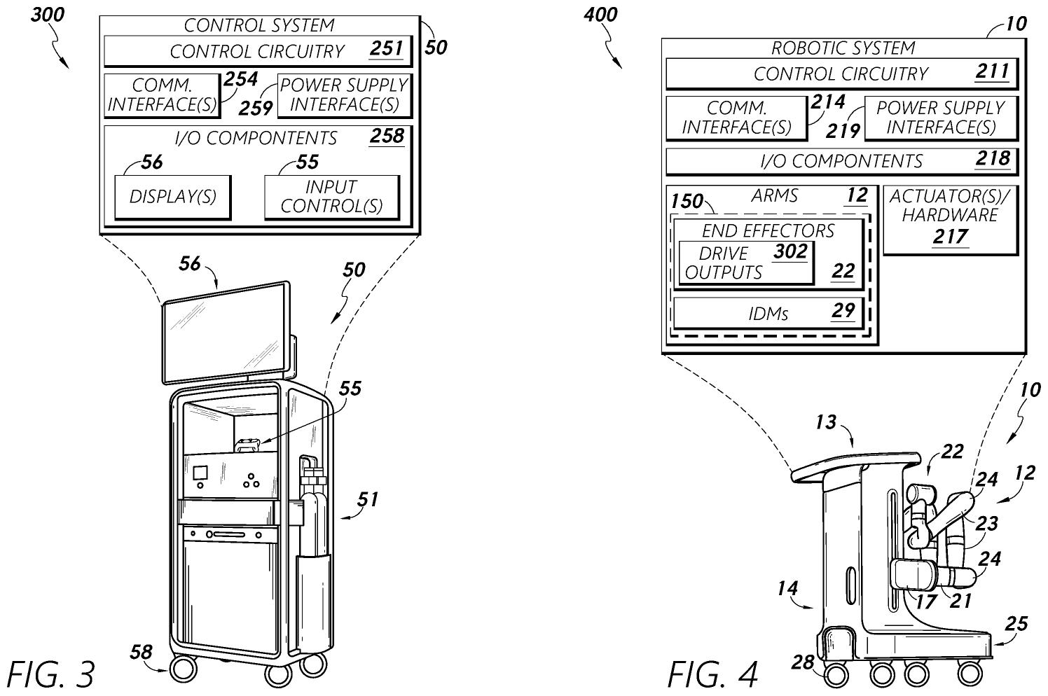

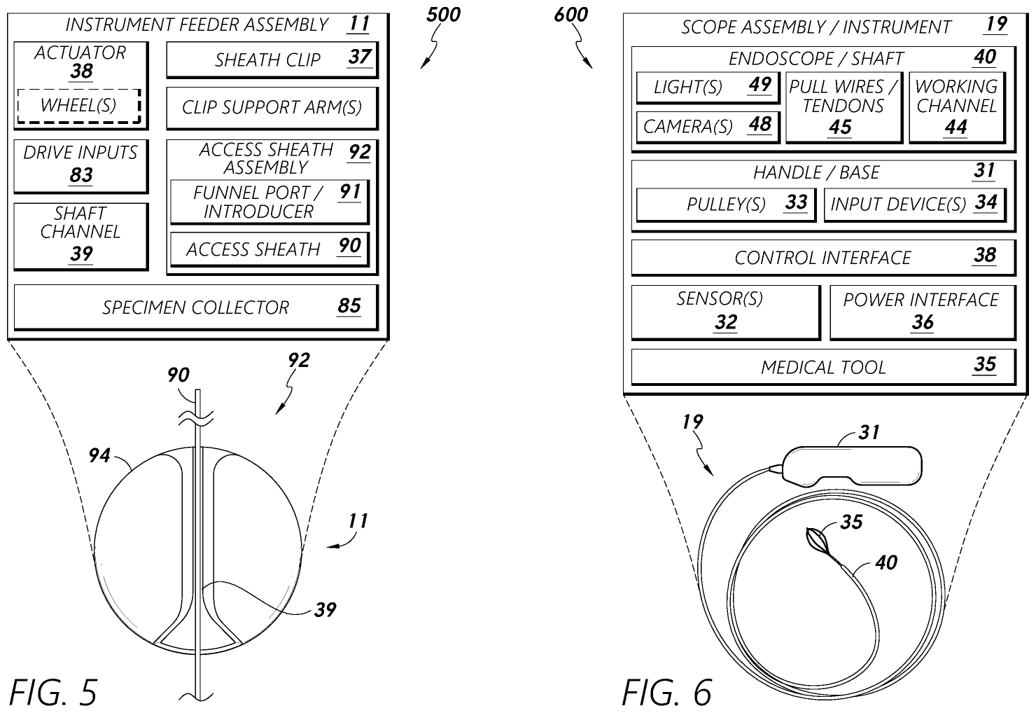

FIG. 3 illustrates an example embodiment 300 of a control system 50 of any system disclosed herein. FIG. 4 illustrates an example embodiment 400 of a robotic system 10 of any system disclosed herein. FIG. 5 illustrates an example embodiment 500 of a robotically-controllable endoscope of any system disclosed herein. FIG. 6 illustrates an example embodiment 600 of a robotic instrument feeder of any system disclosed herein.

With reference to FIGS. 3-6, the control system 50 can be coupled to the robotic system 10 and operate in cooperation therewith to perform a medical procedure. For example, the control system 50 can communicate with the robotic system 10 via a wireless connection or a wired connection (e.g., to control the robotic system 10). Further, in some embodiments, the control system 50 can communicate with the robotic system 10 to receive position data therefrom relating to the position of the distal end of the scope 40. Such positional data relating to the position of the scope 40 may be derived using one or more electromagnetic sensors associated with the respective components, scope image processing functionality, and/or based at least in part on robotic system data (e.g., arm position data, known parameters/dimensions of the various system components, etc.).

The robotic system 10 can be arranged in a variety of ways depending on the particular procedure. The robotic system 10 can include one or more robotic arms 12 configured to engage with and/or control, for example, the scope 40 to perform one or more aspects of a procedure. As shown, each robotic arm 12 can include multiple arm segments 23 coupled to joints 24, which can provide multiple degrees of movement/freedom. When the robotic system 10 is properly positioned, the scope 40 can be inserted into a patient robotically using the robotic arms 12, manually by the physician 5, or a combination thereof. The scope-driver/feeder instrument coupling 11 can be attached to the distal end effector 22 of one of the arms 12b to facilitate robotic control/advancement of the scope 40. Another 12a of the arms may have associated therewith an instrument base/handle 31, wherein the scope 40 is physically coupled to the handle 31 at a proximal end of the scope 40. The scope 40 may include one or more working channels 44 through which additional tools, such as lithotripters, basketing devices, forceps, etc., can be introduced into the treatment site.

The robotic system 10 may be configured to receive control signals from the control system 50 to perform certain operations, such as to position one or more of the robotic arms 12 in a particular manner, manipulate (e.g., advance, articulate) the scope 40, and so on. In response, the robotic system 10 can control, using certain control circuitry 211, actuators 217, and/or other components of the robotic system 10, to perform the operations. For example, the control circuitry 211 may control articulation of the shaft/scope 40 by actuating drive output(s) 302 of the end effector 22 coupled to the instrument handle 31. In some embodiments, the robotic system 10 and/or control system 50 is/are configured to receive images and/or image data from the scope 40 representing internal anatomy of a patient and/or portions of the access sheath or other device components.

The robotic system 10 generally includes an elongated support structure 14 (also referred to as a “column”), a robotic system base 25, and a console 13 at the top of the column 14. The column 14 may include one or more arm supports 17 (also referred to as a “carriage”) for supporting the deployment of the one or more robotic arms 12 (three illustrated in FIGS. 1 and 2). The arm support 17 may include individually configurable arm mounts that rotate along a perpendicular axis to adjust the base of the robotic arms 12 for desired positioning relative to the patient.

The arm support 17 may be configured to vertically translate along the column 14. Vertical translation of the arm support 17 allows the robotic system 10 to adjust the reach of the robotic arms 12 to meet a variety of table heights, patient sizes, and physician preferences. Similarly, the individually configurable arm mounts on the arm support 17 can allow the robotic arm base 21 of robotic arms 12 to be angled in a variety of configurations.

The robotic arms 12 may generally comprise robotic arm bases 21 and end effectors 22, separated by a series of linking arm segments 23 that are connected by a series of joints 24, each joint 24 comprising one or more independent actuators 217. Each actuator may comprise an independently controllable motor. Each independently controllable joint 24 can provide or represent an independent degree of freedom available to the robotic arm.

The robotic system base 25 balances the weight of the column 14, arm support 17, and arms 12 over the floor. Accordingly, the robotic system base 25 may house certain relatively heavier components, such as electronics, motors, power supply 219, communication interfaces 214, I/O components 218, as well as components that selectively enable movement or immobilize the robotic system. For example, the robotic system base 25 can include wheel-shaped casters 28 that allow for the robotic system to easily move around the operating room prior to a procedure.

Positioned at the upper end of column 14, the console 13 can provide both a user interface for receiving user input and a display screen 16 (or a dual-purpose device such as, for example, a touchscreen) to provide the physician/user 5 with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse.

The end effector 22 of each of the robotic arms 12 may comprise, or be configured to have coupled thereto, an instrument device manipulator (IDM) (e.g., instrument base/handle) 11, which may be attached using a sterile adapter component in some instances. The combination of the end effector 22 and associated IDM, as well as any intervening mechanics or couplings (e.g., sterile adapter), can be referred to as a manipulator assembly. In some embodiments, the IDM 11 can be removed and replaced with a different type of IDM, for example, a first type of IDM/instrument may be configured to manipulate an endoscope/shaft, while a second type of IDM/instrument 31 may be associated with the shaft 40 (e.g., coupled to a proximal portion thereof) and configured to articulate the shaft. An IDM can provide power and control interfaces. For example, the interfaces can include connectors to transfer pneumatic pressure, electrical power, electrical signals, and/or optical signals from the robotic arm 12 to the IDM 11. The IDMs 11 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the device manipulators 11 can be attached to respective ones of the robotic arms 12.

As referenced above, the robotic system 10 can include certain control circuitry 211, and further the control system 10 can include control circuitry 251. Any reference herein to control circuitry may refer to circuitry embodied in a robotic system, a control system, or any other component of a medical system. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

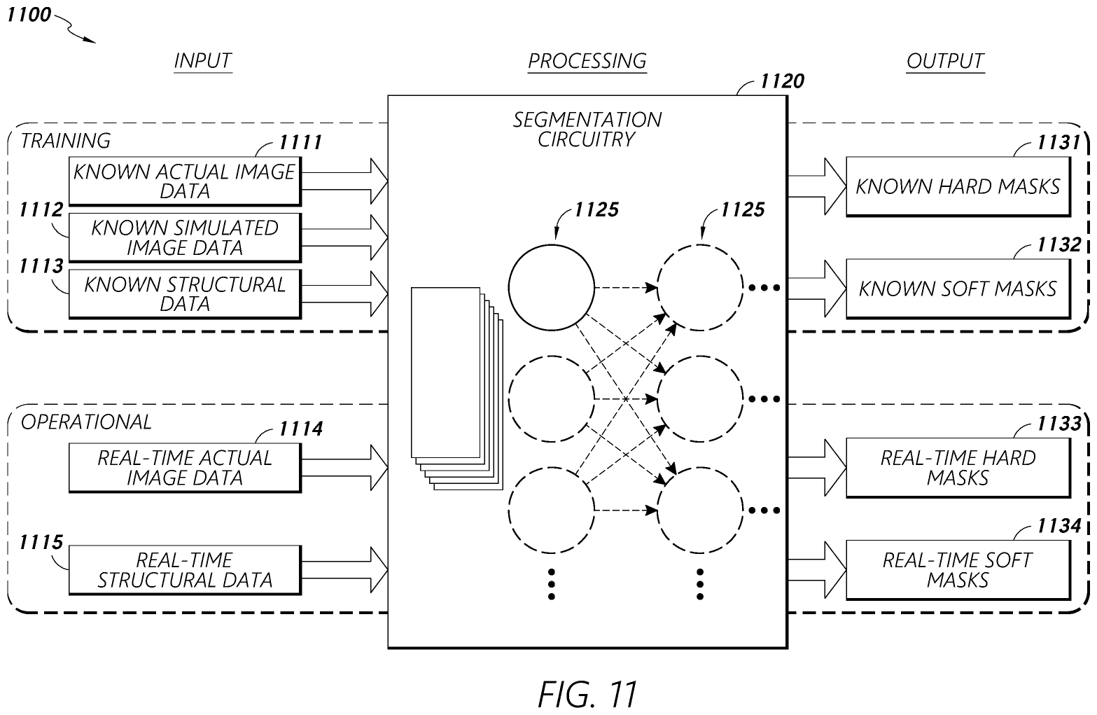

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211,251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein, including the processes shown in FIGS. 11, 15, 26, and 28, as described below.

With respect to the robotic system 10, at least a portion of the control circuitry 211 may be integrated with the base 25, column 14, and/or console 13 of the robotic system 10, and/or another system communicatively coupled to the robotic system 10. With respect to the control system 50, at least a portion of the control circuitry 251 may be integrated with the console base 51 and/or display unit 56 of the control system 50. It should be understood that any description herein of functional control circuitry or associated functionality may be understood to be embodied in the robotic system 10, the control system 50, or any combination thereof, and/or at least in part in one or more other local or remote systems/devices, such as control circuitry associated with a handle/base of a shaft-type instrument (e.g., endoscope) in accordance with any of the disclosed embodiments.

The control system 50 can include various I/O components 258 configured to assist the physician or others in performing a medical procedure. For example, the input/output (I/O) components 258 can be configured to allow for user input to control/navigate the scope 40 and/or other robotically controlled instrument. The control system 50 can include one or more display devices 56 to provide various information regarding a procedure. For example, the display(s) 56 can provide information regarding the scope 40. For example, the control system 50 can receive real-time images that are captured by the scope 40 and display the real-time images via the display(s) 56. Additionally, or alternatively, the control system 50 can receive signals (e.g., analog, digital, electrical, acoustic/sonic, pneumatic, tactile, hydraulic, etc.) from a medical monitor and/or a sensor associated with the patient, and the display(s) 56 can present information regarding the health or environment of the patient.

The various components of the systems of FIGS. 1-6 can be communicatively coupled to each other over a network, which can include a wireless network and/or a wired network. Example networks include one or more personal area networks (PANs), local area networks (LANs), wide area networks (WANs), Internet area networks (IANs), cellular networks, the Internet, personal area networks (PANs), body area network (BANs), etc. In some embodiments, the various communication interfaces 254 can implement a wireless technology such as Bluetooth, Wi-Fi, near-field communication (NFC), or the like. Furthermore, in some embodiments, the various components of the systems can be connected for data communication, fluid exchange, power exchange 259, and so on via one or more support cables, tubes, or the like.

The control system 50 and/or the robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to the respective control circuitry. In some embodiments, the user may engage the user controls 55 to command robotic shaft articulation, as described herein.

With reference to FIG. 5, the instrument feeder assembly 92 can include a channel 39 dimensioned and/or configured for placement therein of at least a portion of a shaft-type instrument, such as an endoscope or the like. For example, when placing a scope or the like to allow for the instrument feeder 11 to axially drive such instrument, the instrument may be nested at least partially within the channel 39. Although illustrated with a channel 39, in some embodiments, instrument feeder devices and assemblies in accordance with aspects of the present disclosure may not include such a channel. the instrument feeder/driver 11 The terms “feeder” and “driver” are used in some contexts herein substantially interchangeably. Therefore, references herein to a scope or instrument feeder can be understood to refer to any type of scope or instrument driver, and vice versa, wherein such devices/systems are configured to actuate, or cause actuation of, a shaft-type instrument in an axial dimension. The actuator 38 may comprise a feed-roller in some embodiments, including any number of roller(s)/wheel(s) configured to effect axial movement of a shaft engaged therewith. The actuator(s) 38 can be controlled through engagement with one or more drive inputs 83, which may allow for physical engagement with mechanical components of the instrument feeder 11 that actuate the actuator means/mechanism 38 and/or may directly actuate the actuator means/mechanism 38.

With reference to FIG. 6, the scope assembly 19 includes a handle or base 31 coupled to an endoscope shaft 40. For example, the endoscope (i.e., “scope” or “shaft”) can include an elongate shaft including one or more lights 49 and one or more cameras or other imaging devices 48. The scope 40 can further include one or more working channels 44, which may run a length of the scope 40. The scope assembly 19 can be powered through a power interface 39 and/or controlled through a control interface 38, each or both of which may interface with a robotic arm/component of the robotic system 10. The scope assembly 19 may further comprise one or more sensors 32, such as pressure sensors and/or other force-reading sensors, which may be configured to generate signals indicating forces experienced at/by one or more components of the scope assembly 19.

The scope assembly 19 includes certain mechanisms for causing the shaft 40 to articulate/deflect with respect to an axis thereof. For example, the shaft 40 may have been associated with a proximal portion thereof, one or more drive inputs 34 associated, and/or integrated with one or more pulleys/spools 33 that are configured to tension/untension pull wires 45 of the scope shaft 40 to cause articulation of the shaft 40.

The scope assembly 19 may be used in conjunction with a medical tool 35 and include various hardware and control components for the medical tool 35 and, in some instances, include the medical tool 35 as part of the scope assembly 19. For example, as shown in FIG. 6, the scope assembly 19 can comprise a basket formed of one or more wire tines. As other examples, the medical tool 35 can be any tool including a radial-probe endobronchial ultrasound (REBUS), ureteral access sheath (UAS), Percutaneous Antegrade Urethral Catheter (PAUC), or the like.

The medical tool 35 and any portions thereof can be powered through a power interface 39 and/or controlled through a control interface 38, each or both of which may interface with a robotic arm/component of the robotic system 10. The scope assembly 19 may use the one or more sensors 32 to sense signals or receive data from the medical tool 35 indicating forces/pressures experienced at/by the medical tool 35. Such sensor readings may be used to determine tool conditions (e.g., stuck basket conditions, capturing of a stone, an opening at an end of an access sheath, or the like), as described in detail herein. In some embodiments, the sensor(s) 32 include one or more sensors configured to directly measure forces are at or near the basket portion 35 of the tines 36.

### Contextual Information Generation Using Multi-Modal Data

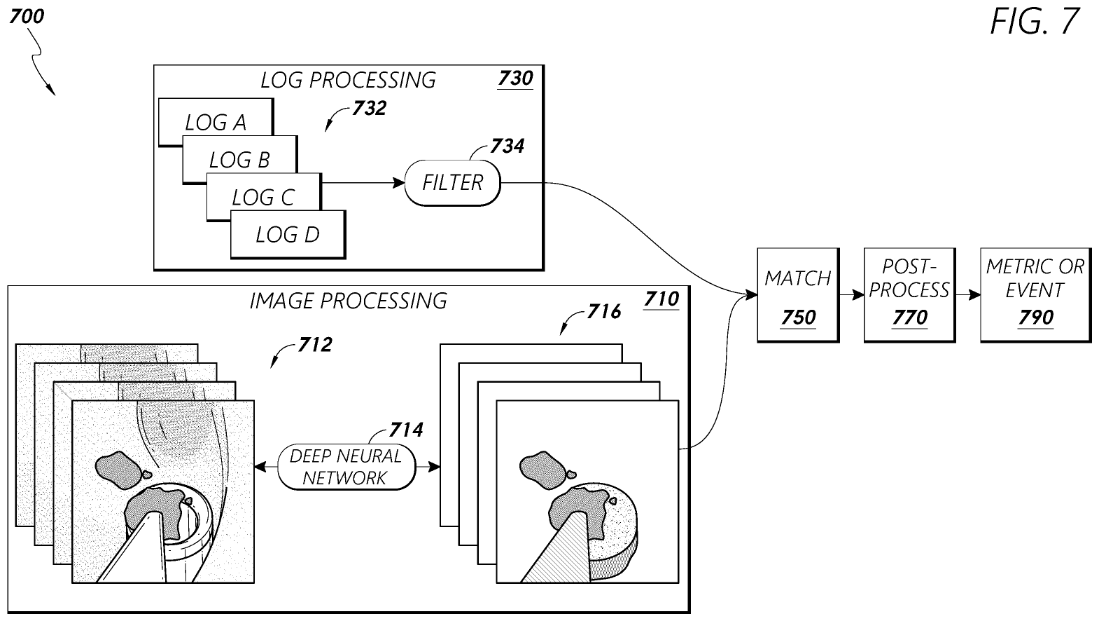

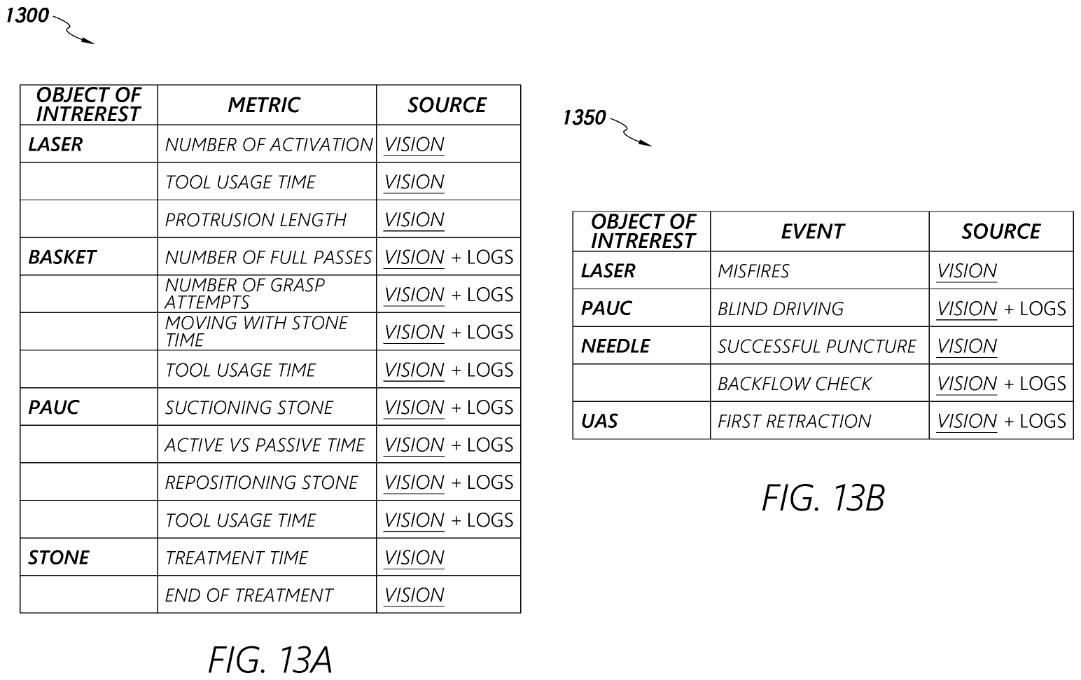

FIG. 7 illustrates an example block diagram of a multi-modal contextual information generator pipeline 700 in accordance with one or more embodiments. As shown, the pipeline 700 can be used to generate metric or event 790 from multi-modal data. Referred in the present disclosure, a metric may be a measurement over a period of time (e.g., a time window) and an event may be an occurrence at a single time in which something happens. Few example metrics and events are listed in relation to FIGS. 13A and 13B.

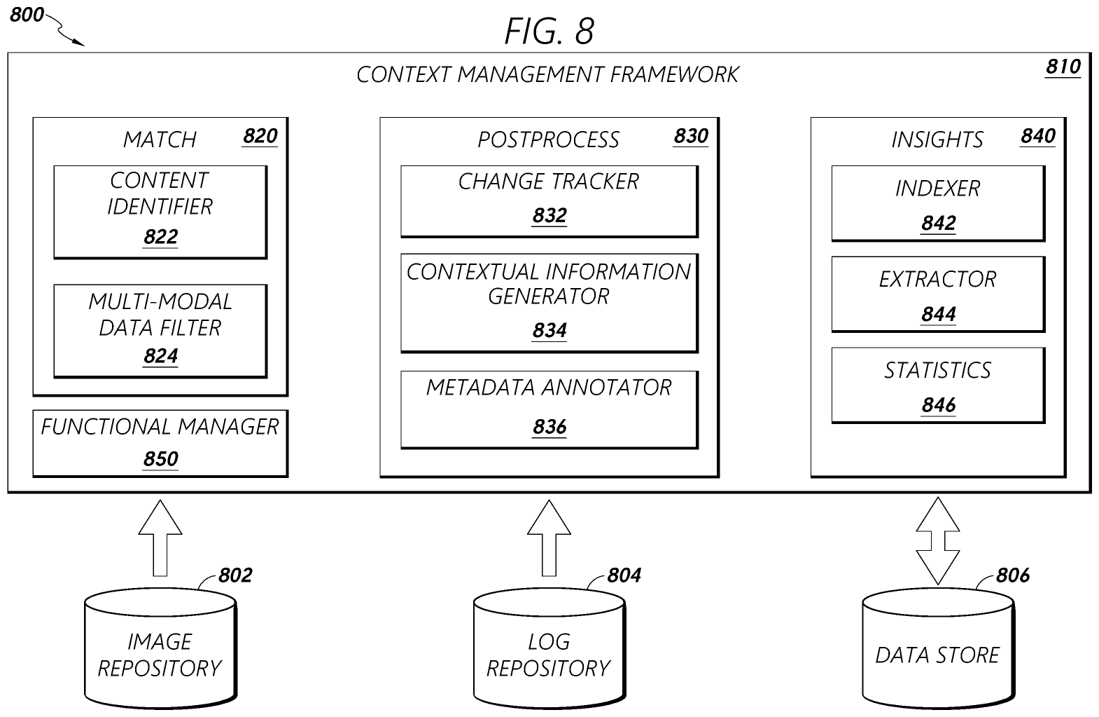

Generally, the pipeline 700 can involve accessing data from two or more modes, such as image data captured via a camera device and log data containing robot data. Such data may undergo preprocessing blocks (e.g., image processing 710, log processing 730) to convert data into more readily computer-analyzable representations. Subsequently, data of a first mode can undergo a matching block 750 with data of a second mode based on one or more matching criteria. Few example matching criteria can include data timestamps, recognized objects, phase or workflow, results, engaged tool functionalities, or the like. The matched data may be undergo a postprocessing block 770 to generate the metric or event 790. Functionalities of each block will be described in greater detail below and in relation to FIG. 8.

As alluded, the pipeline 700 can involve accessing data. The data can be accessed in real-time directly from one or more components of the robotic system or after occurrence from data repositories. For example, image data may be received in real-time as a camera stream (e.g., vision data) from an endoscope or accessed as image frame data from an image repository of past procedures. As another example, robot data may be received in real-time from a control circuitry or from a log repository of past procedures.

Optionally, some accessed data may undergo preprocessing, which may be different for each mode of data, to convert the data into more readily computer-analyzable representations or sizes. For example, image data can be processed into segmented representations. As another example, log data may be processed into subsets, blocks, chunks, or segments of logs based on various filtering criteria.

Referring back to the pipeline 700, image data may undergo image processing 710 involving one or more deep neural network architecture 714 that takes in input image data 712. In some implementations, the input image data 712 may be various image frames taken from a video captured with an endoscopic camera. The deep neural network architecture 714 can be configured to generate output representations 716 that indicate which pixels and regions in an image frame belong to the same object or share similar characteristics (e.g., object segmentation). In some instances, the deep neural network architecture 714 may include machine learning classifiers may process the output representations 716 to identify, for example, various objects including medical tools, stone(s), anatomical features, etc (e.g., object recognition). In some instances, the output representations 716 can be further processed to readily provide information in connection with presence, position, and orientation of the objects.

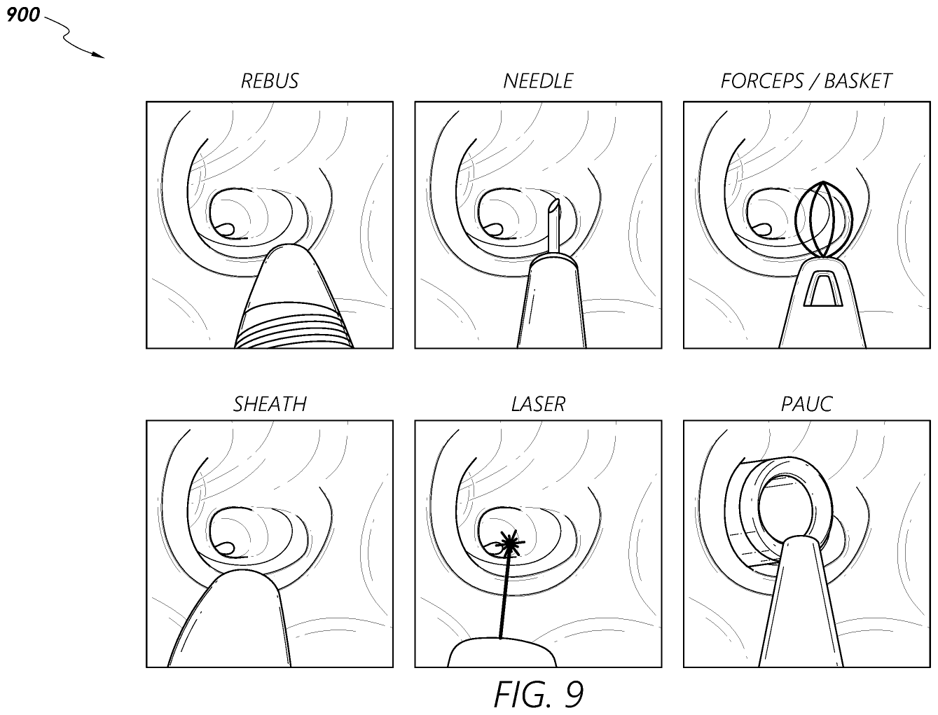

In some embodiments, the deep neural network architecture 714 can be configured to capture the features of the images and incorporate temporal information. These embodiments can use Convolutional Neural Networks (CNNs) to extract and capture features of the images, which may be followed by Recurrent Neural Networks (RNNs) such as Long-short term memories (LSTMs), to capture the temporal information and sequential nature of the activities. Temporal Convolutional Networks (TCNs) are another class of architectures that can be used for surgical phase and activity recognition, which can perform predictions that are more hierarchical and retain memory over an entire procedure (as opposed to LSTMs which retain memory for a limited sequence and process temporal information in a sequential way). The deep neural network architecture 714 may facilitate tool identification and motion detection, which are described with greater detail with respect to FIG. 9 to identify its example classifications 900.

Data other than vision data may undergo log processing 730 involving selecting a subset of log data 732 using one or more filter(s) 734. For instance, the filter(s) 734 can be applied the log data 732 to select only robot data pertaining to command data, control state, and/or success status in connection with a needle tool.

The log data 732 can include system logs including command data automatically generated or provided via user input, state data including kinematic state data, tool status including connection status, or the like. Additionally, system logs may include any annotations or metadata. For example, user interactions with a user interface may provide valuable information about phases/workflow steps and notes taken by physicians and entered into the system logs may provide additional detail to what is observed (e.g., steps taken, tools engaged, results, or the like) during a medical procedure. In some embodiments, the log data 732 may include voice recordings of physicians performing or providing contextual information in connection with the medical procedure. The voice recordings may be raw or parsed with natural language models and associated with timestamps to be matched as multi-modal data. The log data 732 can include other logs such as sensor data (e.g., EM data, torque data) and derived tool information to be used as multi-modal data.

The matching block 750 and the postprocess block 770 may depend on metrics or events 790 of interest. Some metrics or events 790 may rely on data of a single mode and may skip the matching block 750. For example, it may be possible to determine a needle “successful needle puncture” event, as described in FIG. 13B, based on image processing 710 without log processing 730. That is, the postprocess block 770 can involve only confirming visual of a needle in an image frame for the event. In contrast, some metrics or events 790 may rely on multi-modal data. For example, a basket “moving with the stone time” metric of FIG. 13A relies on both the image processing 710 and the log processing 730 to find a union/intersection between when a stone is detected and when the basket moves to determine the duration metric.

For the matching block 750, any matching criteria may be used to match data from two sources to determine multi-modal data. In the example of the “moving with the stone time” metric above, the duration metric may be determined based on timestamps associated with image frames depicting the stone. Relying on the timestamps, robot data in the log processing 730 can be filtered to identify a subset of the robot data having the same timestamps. Some other metrics or events 790 may instead rely on log data to filter image data. For example, attempted stone capture attempts may be identified from the log processing 730 and a subset of video can be selected based on command timestamps associated with the basket open/close commands. In some embodiments, the matching of multi-modal data may be based on operational data including phases or workflow of medical procedures, results thereof, engaged tool functionalities, or the like. The matching block 750 will be described in greater detail below with reference to a match module 820 of FIG. 8.

The postprocessing block 770 can follow the matching block 750 to generate contextual information, which include the metrics and events 790. In contrast with just image information in image data, additional data of another mode can help determine context of what is happening to the image information. The postprocessing block 770 will be described in greater detail below with reference to a postprocess module 830 of FIG. 8.

It is noted that the pipeline 700 may be executed to determine the metrics and events 790 as a push process or as a pull process. Regarding the push process, the pipeline 700 may be executed to determine all or substantially all metrics and events 790 in anticipation of future access. In a sense, the push process works best with static data (e.g., data that substantially does not get updated frequently) and can perform the pipeline 700 as a batch process for the entirety of a video. Regarding the pull process, the pipeline 700 may be executed to update any metrics or events 790 that are affected by newly acquired data. For example, if newly acquired vision data depicts a LASER, then metrics and events 790 pertaining to the LASER may be selectably updated. Accordingly, the pull process is more desirable for online, real-time metrics and events 790. In some embodiments, the pipeline 700 may be implemented as a combination of the push process and the pull process.

### Context Management Framework

FIG. 8 illustrates an example system 800 including a context management framework 810 in accordance with one or more embodiments. The context management framework 810 can be configured to determine and provide contextual information, such as metrics and events (e.g., the metrics and events 790 of FIG. 7), and provide various functionalities based on the contextual information. For example, the context management framework 810 can determine a context relevant to a target metric or event, access and select one or more segments of multi-modal based on the determined context, and determine the target metric or event using the segments of multi-modal data. The image data can be indexed and queried based on the contextual information for ready access. Some functionalities of the robotic system may be automatically enabled or disabled based on the contextual information.

As shown, the context management framework 810 can include a match module 820, a postprocess module 830, an insights module 840, and a functional manager module 850. It should be noted that the components (e.g., modules) shown in this figure and all figures herein are exemplary only, and other implementations may include additional, fewer, integrated or different components. Some components may not be shown so as not to obscure relevant details.

In some embodiments, the various modules and/or applications described herein can be implemented, in part or in whole, as software, hardware, or any combination thereof. In general, a module and/or an application, as discussed herein, can be associated with software, hardware, or any combination thereof. In some implementations, one or more functions, tasks, and/or operations of modules and/or applications can be carried out or performed by software routines, software processes, hardware, and/or any combination thereof. In some cases, the various modules and/or applications described herein can be implemented, in part or in whole, as software running on one or more computing devices or systems, such as on a user or client computing device, on a server, or a control circuitry (e.g., the control circuitry 211, 251 of FIGS. 3 and 4). For example, one or more modules and/or applications described herein, or at least a portion thereof, can be implemented as or within an application (e.g., app), a program, or an applet, etc., running on a user computing device or a client computing system. In another example, one or more modules and/or applications, or at least a portion thereof, can be implemented using one or more computing devices or systems that include one or more servers, such as network servers or cloud servers. It should be understood that there can be many variations or other possibilities.

As shown with the example system 800, the context management framework 810 can be configured to communicate with one or more data repositories (e.g., an image repository 802, a log repository 804, etc.). Each of the data repositories can be configured to store and maintain various types of data to support the functionality of the context management framework 810. For example, the image repository 802 may store image data including video/vision data consistent with data accessed in the image processing 710 of FIG. 7. As another example, the log repository 804 may store log data including robot data and sensor data consistent with data accessed in the log processing 730 of FIG. 7. In addition to the image data and the log data, the data repositories may store supplementary data including procedure identifiers (e.g., phase, workflow, etc.), operation identifiers, patient identifier, timestamps, metadata, attributes, system time, relational database identifiers, and other information required by the context management framework 810. It is noted that some image data or log data may be real-time data and may be made available directly from the robotic medical system without having to access the data repositories.

The match module 820 can be configured to identify relevant context and select multi-modal data. In connection with these functionalities, the match module 820 can include a context identifier module 822 and a multi-modal data filter module 824.

The context identifier module 822 can, for target contextual information (e.g., a target metric or event), determine (i) one or more medical tools relevant for the contextual information and (ii) one or more data sources from which to access multi-modal data. As an example metric, the context identifier module 822 can determine (i) that a basket tool is relevant for the “number of grasp attempts” metric from FIG. 13A and (ii) that robot data reflecting opening and closing of the basket tool as well as image data confirming successful or failed attempts should be accessed. As an example event, the context identifier module 822 can determine (i) that a LASER tool is relevant for “misfires” event from FIG. 13B and (ii) that only image data is to be accessed.

The multi-modal data filter module 824 can filter one or more portions or segments of data relevant for the target contextual information. When the target contextual information includes metrics or events involving a particular object or a medical tool, segments of image data depicting the object or the medical tool may be filtered. In some instances, the segments may be filtered based on object recognition or object criteria. For example, image data segments depicting a stone can be filtered based on recognition of a stone for the “treatment time” metric of FIG. 13A. Similarly, the filtered image data segments may be further filtered based on additional recognition of a basket for the “number of grasp attempts” metric of FIG. 13B. In some embodiments, segments may be defined by one or more timestamps (e.g., a time window) and such timestamps can be used to filter (e.g., filter) data of another mode. In some embodiments, data segments may be filtered based on identifiers (e.g., phase identifier, result identifier, tool identifier, etc.). It is understood that two or more filters can be used in combination or in sequence to select unions or intersections of multi-modal data segments.

The postprocess module 830 can be configured to generate the contextual information and, optionally, annotate original data with metadata that describe characteristics, properties, or context of the original data. In connection with these functionalities, the postprocess module 830 can include a change tracker module 832, a contextual information generator module 834, and a metadata annotator module 836.

The change tracker module 832 can track changes (e.g., generate change data) from the segments of multi-modal data. For example, where the segments contain sequential vision image data, various vision-based techniques such as optical flow techniques may analyze the displacement and translation of image pixels in a video sequence in the vision image data to infer camera movement as the tracked change. Examples of optical flow techniques may include motion detection, object segmentation calculations, luminance, motion compensated encoding, stereo disparity measurement, etc. The optical flow technique may generate change data reflecting the tracked change. As another example, robot data logging one or more cycles of insertion/retraction based on insertion length or open/close of a basket tool can be tracked with change data reflecting a number of passes and grasp attempts. As additional examples, sensor data logging positions of a scope tip determined with EM sensors may be tracked with change data reflecting trajectories and force measured on a torque sensor may be tracked with change data based on when the sensed force is greater than a threshold level.

The contextual information generator module 834 can generate the metrics and events using the segments of multi-modal data. Process involved for the determination of the contextual information may depend on target contextual information. For example, each target metric or target event may involve a process that has a separate and distinct set of input data and processing of the data. In some instances, the process may determine the target contextual information using the tracked changes (e.g., based on the change data) provided by the change tracker module 832. Various example metrics and events are respectively presented in FIG. 13A and FIG. 13B and relevant processes involved for the metrics and events will be described in greater detail there. The metrics and events could be used as the basis for other outputs and features. For example, detection of “blind driving” event can serve to provide a warning notification to a display without interfering with a medical procedure. As another example, treatment time metric may be determined as aggregation of other tool usage metrics.

The metadata annotator module 836 can be configured to generate and annotate (e.g., add or associate) any data with metadata. Metadata as described herein can refer to any data that provides information, including contextual information, about other data. As few examples, metadata can include the metrics and events, phases/workflow of a medical procedure, results (e.g., successful, unsuccessful, completion percentage, etc.), voice recordings (e.g., raw or parsed), warnings, identifiers, or the like. In some embodiments, the metadata may help segment any data, such as video data, such that each segment may be indexed. For example, the metadata can include a timestamp associated with one or more image frames of the video data that help categorize the image frames. As another example, the metadata can include any identifiers (e.g., the phases/workflow, results, parsed recordings, etc.) such that any data may be indexed based on the identifiers. As will be described further below, the metadata can help improve various functionalities of an insights module 840.

As shown in FIG. 8, the context management framework 810 can be configured to communicate with a data store 806. The data store 806 can be configured to store and maintain various data types to support the functionality of the content management framework 810. For example, the data store 806 can store metrics and events, other contextual information, metadata, annotations, statistics, and other information required by the context management framework 810. Some of the data stored may be determined/generated online (e.g., during operation of a medical procedure) or may be determined/generated offline (e.g., after the medical procedure is completed) based on collected data. For example, a needle “successful puncture” event may be determined/generated in real-time during operation or, alternatively, determined/generated after the operation based on analysis of image data or log data. In some embodiments, it is also contemplated that some data may not be stored in the data store 806 but may be determined/generated on as-requested basis and discarded once consumed.

The insights module 840 can be configured to provide search functionalities (e.g., queries, filter, sort, indexing, access, etc.), extraction of data segments, and various statistics. In connection with these functionalities, the insights module 840 can include an indexer module 842, an extractor module 844, and a statistics module 846. Some or all of the functionalities of the insights module 840 may be presented to a physician via a display and be controlled via interface elements.

The indexer module 842 can be configured to receive a query containing one or more search criteria, access the data store 806, filter data based on the search criteria, and provide results. The search criteria can include any metrics or events, metadata, annotation, statistics, structural data, or other information and the query may include comparative terms. For example, a physician may instruct the indexer module 842 with a query requesting all instances when a LASER “tool usage time” metric exceeds 0.5 seconds or where a PAUC “blind driving” event is detected. In some implementations, the indexer module 842 may provide a physician with options of additional filtering or sorting of the results. For example, the physician may instruct the indexer module 842 to provide LASER “tool usage time” metrics exceeding 0.5 seconds in a descending order or with a follow-up query requesting any of the results also includes a LASER “misfires” event. Similarly, other queries may ask the indexer module 842 with queries requesting a particular phase, a certain result of a medical procedure, or annotation (e.g., parsed voice recording) that is synonymous with “stone retrieval.”

The extractor module 844 can be configured to extract one or more segments of original data for review. Specifically, the extractor module 844 may extract image frames of vision data from the image repository 802 and provide the image frames as a sequence in association with playback controls. The extraction can be based on user selection or based on search results of the indexer module 842. For example, the extractor module 844 may provide a set of sequential image frames showing PAUC “repositioning stone” metric or showing LASER “misfire” event. In some embodiments, the extractor module 844 may be configured to provide data relied on, which may be one or more segments of multi-modal data, to determine/generate contextual information. For example, the extractor module 844 may extract and provide image data and log data relied on for a determination of a basket “number of full passes” metric.

Accordingly, the queries of the indexer module 842 and the extracted image frames of the extractor module 844 can enable the insights module 840 to be used as a case indexing tool. The case indexing tool can enable easy navigation of case videos and find when a specific tool is in view and/or being in use. Combined with the metrics and events determined by the contextual information generator module 834, the case indexing tool can also provide navigation to parts of the case where a certain metric or event is found. For example, physicians can use the case indexing tool to jump to a specific part of a video and corresponding logs rather than having to watch the entire case of interest. Similarly, the physicians may review their performance on a tool or capture segments as a demonstration for training new users. For example, providing the last 10 examples of successful basketing for a new user. Additionally, the case indexing tool can be helpful to engineers who are working to improve the robotic medical system. For example, engineers who are working with a basket exhibiting repeated failures of grasp attempts can easily filter for cases with high number of repeated grasp attempts.

The statistics module 846 can be configured to determine and provide various statistics. The statistics can include descriptive measures including mean, median, mode, standard deviations, or the like. For instance, average tool usage time may be determined and provided. The statistics module 846 may additionally determine and provide inferential measures including confidence intervals, regression analysis, variance analysis, or the like. In some embodiments, the statistics module 846 may be configured to communicate with the indexer module 842 and the extractor module 844 to provide searching and extraction features. For example, a physician may inquire video segments where a frequency of basket “number of grasp attempts” metric was in the 90% percentile and the statistics module 846 may work in connection with the indexer module 842 and/or the extractor module 844 to provide the video segments. Statistics determined by the statistics module 846 may be stored in the data store 806 to provide cached access.

The functional manager module 850 can be configured to control various functionalities of the robotic medical system 100 of FIG. 1. In some embodiments, based on contextual information, such as the metrics or events, certain functionalities of the robotic medical system or any portions there of (e.g., medical instrument, medical tool, scope/shaft, or the like) may be enabled or disabled. For example, “fast insertion/retraction” feature that may speed up insertion and retraction of the scope/shaft can be enabled when UAS “fast retraction” event is detected. The speeding up of the scope/shaft control may reduce operation time and improve user experience. When the “fast retraction” event is not detected, the feature can be disabled. In some implementations, the controlling of a feature may not be binary (e.g., enable or disable) but may be gradual with variable increase or decrease in speed. The functional manager module 850 can automate surveying of current context and determination of when to allow the use of such features. In some implementations, the functional manager module 850 may provide warnings in connection with the context, which may include the metrics and events, or provide prompts in connection with use of such features.

### Medical Tool Identification

FIG. 9 illustrates example classifications 900 of various medical tools (e.g., the medical tool 35 of FIG. 6). The example classifications 900 show a REBUS, a needle, forceps/basket, a sheath (e.g., a UAS), a LASER, and a PAUC, but it is to be understood that the classifications can include various other medical tools. After identifying a portion/region of image data associated with the medical tool, additional image processing can be performed (e.g., using machine learning algorithms, selection criteria, etc.) to identify the medical tool at the portion/region. In some embodiments, one or more classifiers (e.g., the deep neural network architecture 714 of the image processing 710 of FIG. 7) can be configured to provide the tool identification.

Supplemental data from the robotic system performing a medical procedure, such as bronchoscopy, can be used to aid in tool identification. Such supplemental data may include phase information for the procedure, which can be used to narrow down the possible medical tools based on knowledge of the typical tools used during particular phases of the bronchoscopy procedure. For example, during a targeting phase and biopsy phase, the tools likely used are REBUS, needle, and forceps/basket. If the bronchoscopy procedure Is in those phases, then the possible choices for the tool identification for the tool recorded in a video can be narrowed down to those possibilities.

In addition, vision data of the medical procedure (e.g., bronchoscopy video captured by an endoscope) can be analyzed to identify the motion of the medical tool tracked in the video frames.

In one example, a REBUS can be identified by looking for a specific motion. A REBUS is typically used to get confirmation of a nodule location. One type of REBUS has a tip of that is silver with ridges. The ridges may form a spiral or screw around the surface. During use, movement of the REBUS can include rotation. This rotation is captured across several frames of the video and can be identified in the video, for example, by tracking the movement of the ridges. This rotation motion can be used to identify a tracked medical tool used during the targeting/biopsy phase as a REBUS.

In another example, a needle can be identified by looking for a specific motion. The needle is typically used to get a biopsy sample once a nodule is localized. During sampling, the needle typically moves in a back and forth dithering motion. This dithering motion can be used to identify a tracked medical tool used during the targeting/biopsy phase as a needle.

In another example, forceps/basket can be identified by looking for a specific motion. The motion can include a quick and hard pull motion, as the forceps/basket are used to pull a sample from lung tissue. This pulling motion can be used to identify a tracked medical tool used during the targeting/biopsy phase as forceps/basket.

Furthermore, there may be sensor data or robot data available from the robotic medical system that can further narrow down the possible medical tool. For example, sensors in the robotic system may be able to identify the change in position and orientation imparted on the medical tool being manipulated by the robotic medical system based on EM sensor data or force/pressure expected during a stone removal based on torque sensor data. Similarly, robot command data (e.g., LASER “fire” command) or robot state data (e.g., kinematic data including insertion length and scope/shaft 40 position and orientation) may facilitate the tool identification. Different embodiments may use different types of classifiers or combinations of classifiers. In some embodiments, sequence based models that try to capture the temporal information and sequence of activities in a procedure may additionally provide identification of surgical activity. For instance, biopsy activity and stone removal activity may be identified based on the above described motions in relation to the relevant tools. In some implementations, identified activities may be categorized in a sequential manner (e.g., a first phase or a second phase of a workflow) or hierarchical manner (e.g., phases/tasks, activities/sub-tasks, etc.)

### Labelling Masks: Hard Masks and Soft Masks

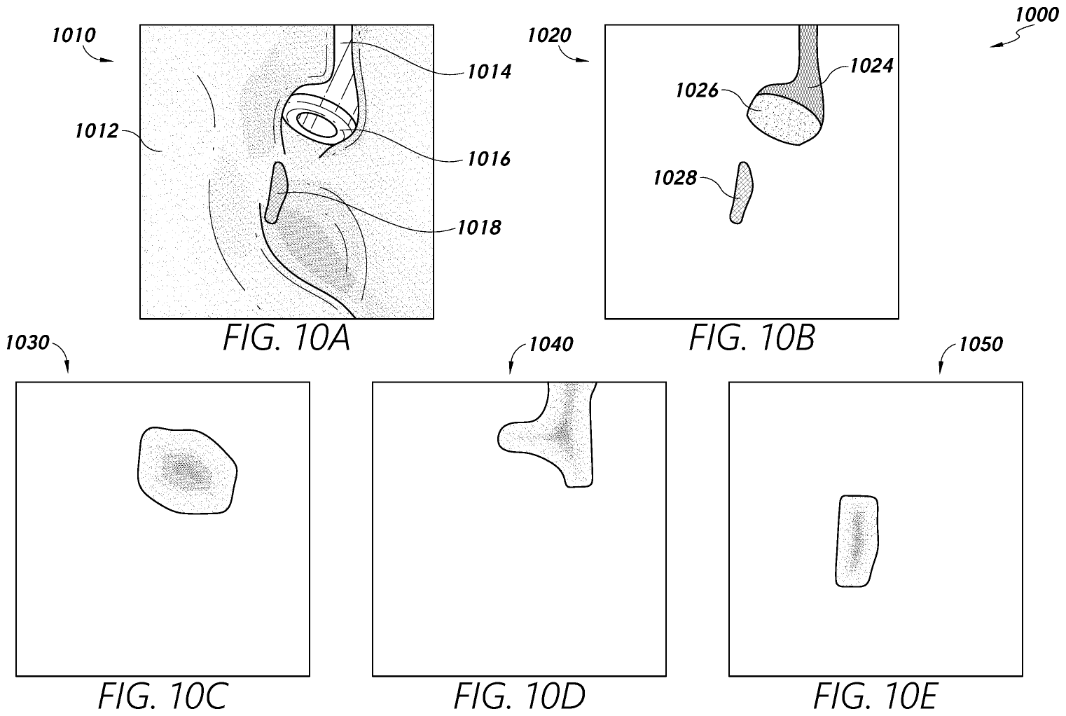

FIG. 10 illustrates hard and soft masks 1000 usable in labelling of various objects in accordance with one or more embodiments. Specifically, FIG. 10A illustrates an example captured image 1010 (e.g., the input image data 712 of FIG. 7, which may be vision data captured from the scope/shaft 40 of FIG. 6). The captured image 1010 illustrates various objects and portions thereof including a PAUC (a sheath 1014 and a tip 1016) and a stone 1018 against a background 1012.

A mask, in the context of semantic segmentation, can represent boundaries of an object. Various neutral network architecture can be trained based on the captured images and the masks to infer presence and boundary of the object in newly captured images. The mask may be a hard mask or a soft mask, either of which may be generated from image data via image processing. For example, an example hard mask 1020 of FIG. 10B and example soft masks 1030, 1040, 1050 of FIGS. 10C-10E can be generated with the image processing 710 of FIG. 7 from the captured image 1010.

In the example hard mask 1020, objects of interest in the captured image 1010 is labeled with masks drawn around the objects. The masks are considered hard masks (also referred as hard labels) in that each mask drawn (i.e. every pixel in the mask) is of a single object and everything outside of the mask is not the object. For example, a sheath hard mask 1024 provides a boundary for the PAUC sheath 1014, a tip hard mask 1026 for the PAUC tip 1016, and a stone hard mask 1028 for the stone 1018. Each hard mask of an object can be color coded with a unique color.

The example soft masks 1030, 1040, 1050 each illustrates a corresponding mask for the PAUC sheath 1014, PAUC tip 1016, and the stone 1018, A soft mask (also referred as a soft label) is contrasted from a hard mask in that the soft mask does not provide a binary determination of whether a pixel belongs to an object or not but bases its determination on a spectrum. For instance, pixels closer to the center of the soft mask may be considered more likely to be the object and, on the contrary, pixels further away from the center of the mask may be considered to be less likely to be the object. A coding scheme, such as a coloring scheme, may be utilized to show the spectrum. For example, yellow can represent pixels that are 100% the object of interest while purple can represent pixels that are 0% with every color in between representing intermediate likelihoods on the spectrum. In some implementations, the pixels can be coded based on confidence values assigned to respective pixels that indicate a network's (e.g., the deep neural network architecture 714 of FIG. 7) confidence in classifying the pixel as a part of an object of interest. Soft mask implementations can be particularly useful for objects whose boundaries are ambiguous and therefore difficult to precisely mark with hard masks.

In some embodiments, each mask may relate to one object of interest (e.g., the soft masks 1030, 1040, 1050) and suck individual masking may provide additional utility. For instance, each mask may be individually relaxed so as to not include a corresponding object. Additionally, each mask can provide versatility to be transformed to other forms of labels as desired. For instance, the mask can be changed to a bounding box if spatial information with respect to a camera is desired. Furthermore, one or more keypoints may be derived from the area of the mask, including calculation of geometric points like the center of mass.

### Object Segmentation Framework and Training Data Generation

FIG. 11 illustrates an image-based object segmentation framework 1100 in accordance with one or more embodiments. The object segmentation framework 1100 can be configured to predict masks used for segmenting image data. The object segmentation framework 1100 may be embodied in certain control circuitry, including one or more processors, data storage devices, connectivity features, substrates, passive and/or active hardware circuit devices, chips/dies, and/or the like. For example, the object segmentation framework 1100 may be embodied in any of the control circuitry 251, 211 shown in FIGS. 3 and 4 and described above. The object segmentation framework 1100 may employ machine learning functionality to perform object segmentation on, for example, endoscopic images captured during a medical procedure.

The object segmentation framework 1100 may be configured to operate on certain image-type data structures, such as image data representing at least a portion of a treatment site associated with medical procedure(s). Such input data/data-structures may be operated on in some manner by certain segmentation circuitry 1120 associated with an image processing portion of the object segmentation framework 1100. The segmentation circuitry 1120 may comprise any suitable or desirable segmentation architecture, such as any suitable or desirable artificial neural network architecture.

The segmentation circuitry 1120 may be trained according to input image data and output representations corresponding to the respective image data as input/output pairs, wherein the segmentation circuitry 1120 is configured to adjust parameters or weights (e.g., neurons 1125) associated therewith to correlate the input image data to the output representations. The image data as input to the segmentation circuitry 1120 can comprise video or still images. The image data can include known actual image data 1111 or known simulated image data 1112 and the representations can include the known hard masks 1131 or the known soft masks 1134. The input image data, the segmentation circuitry 1120, and the output representation may respectively correspond to the input image data 712, the deep neural network architecture 714, and the output representation 716 of the image processing 710 of FIG. 7. The segmentation circuitry 1120 (e.g., convolutional neural network) may be trained using a labelled dataset and/or machine learning. The object segmentation framework 1100 may be configured to execute the learning/training in any suitable or desirable manner.

In some implementations, instead of the known actual image data 1111, the segmentation circuitry 1120 may be trained based on known simulated image data 1112. For example, the known simulated image data 1112 may be generated with data generation models and used as additional training data. For example, Generative Adversarial Networks (GANs) are neural network models that learn to generate images by having two image datasets from two domains. Here, a first domain can include datasets containing the known actual image data 1111 and a second domain can include simulated datasets containing the known simulated image data 1112 generated with a Generator of a GAN. A Discriminator of the GAN can be trained to distinguish a real image (e.g., the known actual image data 1111) and a synthetic image (e.g., generated by the Generator, the known simulated image data 1112). The Generator works to fool the Discriminator and the Discriminator works to correctly sort real images from synthetic images. After sufficient training of the GAN model, the known simulated image data 1112 in the second domain can be treated as the known actual image data 1111 in the first domain to increase availability and size of training dataset. The larger training dataset can help get rid of artifacts and mismatches between the masks and the background.

In some implementations, shape constraints can be applied to the data generation model when training to help create more realistic generated images. The masks provided by the segmentation circuitry 1120 can provide a good idea of how each object of interest (e.g., a stone, PAUC tip, PAUC sheath, etc.) with their shapes and the shape information can be injected into the training process of the data generation model. For example, the Generator/Discriminator of the GAN can limit its generation and identification using the shape information.

The known hard masks 1131 and the known soft masks 1132 may be generated at least in part by manually labeling anatomical features in the known actual image data 1111. For example, manual masks may be determined and/or applied by a relevant medical expert to segment which medical tool is where in images captured by an endoscope. The known input/output pairs can indicate the parameters of the segmentation circuitry 1120, which may be dynamically updatable in some embodiments. In some implementations, known structural data 1113 may further be used to train the segmentation circuitry 1120 to produce segmentation masks (e.g., the known hard masks 1131 and the known soft masks 1132).

The known structural data 1113 can include additional data provided to the segmentation circuitry 1120 that can facilitate contrastive learning. Contrastive learning is an approach to learning that focuses on extracting meaningful representations by contrasting positive and negative pairs of instances. Importantly, contrastive learning leverages the assumption that similar instances should be closer together in a learned embedding space while dissimilar instances should be farther apart in the space. The known structural data 1113 can facilitate contrastive learning by structurally categorizing/indexing actual and simulated images of a medical image domain. The categorizing/indexing can help quantify expected similarity and dissimilarity between two or more images.

The known structural data 1113 can contain phases, clinical workflow steps, tool identification, labels, or any other data that can be associated with the input image data to sort the input image data into a data structure. In some implementations, the known structural data 1113 may be an output of a separate model other than the segmentation circuitry 1120 that determines the known structural data 1113 for the input image data. Using the structure that differentiates/organizes/categorizes/sorts the input image data, contrastive learning can create specialized encoders for the segmentation circuitry 1120 in the medical image domain by learning a representation of images that separates members of one structure from members of another structure, where members refer to images or representations thereof. The encoder learns from images in the same image domain as the tasks solved and, thus, can provide a more compact and effective segmentation circuitry 1120.

In some embodiments, the object segmentation framework 1100 may be configured to generate real-time hard masks 1133 and/or real-time soft masks 1134 as inferences of the segmentation circuitry 1120 using the parameters or weights (e.g., neurons 1125) adjusted during the training. Example hard masks 1133 and soft masks 1134 were described in relation to FIGS. 10B-10E. The real-time hard masks 1133 and/or the real-time soft masks 1134 can provide segmented representations of real-time actual image data 1114. In some embodiments, the segmentation circuitry 1120 may take as input real-time structural data 1115 that is similarly structured with the known structural data 1113 used for the contrastive learning.

The segmentation circuitry 1120 may include a plurality of neurons (e.g., layers of neurons 1125, as shown in FIG. 11) corresponding to overlapping regions of an input image that cover the visual area of the input image. The segmentation circuitry 1120 may further operate to flatten the input image, or portion(s) thereof, in some manner. The segmentation circuitry 1120 may be configured to capture spatial and/or temporal dependencies in the input images through the application of certain filters. Such filters may be executed in various convolution operations to achieve the desired output data. Such convolution operations may be used to extract features, such as edges, contours, and the like. The segmentation circuitry 1120 may include any number of convolutional layers, wherein more layers may provide for identification of higher-level features. The segmentation circuitry 1120 may further include one or more pooling layers, which may be configured to reduce the spatial size of convolved features, which may be useful for extracting features which are rotational and/or positional invariant, as with certain anatomical features. Once prepared through flattening, pooling, and/or other processes, the image data may be processed by a multi-level perceptron and/or a feed-forward neural network. Furthermore, backpropagation may be applied to each iteration of training. The segmentation circuitry 1120 may able to distinguish between dominating and certain low-level features in the input images and classify them using any suitable or desirable technique. In some embodiments, the neural network architecture of the segmentation circuitry 1120 can comprise any of the following known convolutional neural network architectures: LeNet, AlexNet, VGGNet, GoogLeNet, ResNet, ZFNet, or the like. In some embodiments, the neutral network can be an implementation of an encoder-decoder architecture with a pre-trained encoder with skip connections, such as AlbUnet. AlbUnet is a U-Net with ResNet encoders and, in the present disclosure, can take image data as input and output segmented image where each pixel is labeled.

The segmentation circuitry 1120 may employ more than one type of machine learning algorithm (e.g., UNet, AlbUNet, MaskRCNN, etc.) to perform segmentation and to generate a mask identifying portions of an input image comprising an object. In some embodiments, results from the various machine learning algorithms may be combined to generate the mask. In some cases, particular machine learning algorithms may be better at segmenting certain types of objects. For instance, one type of machine learning may be better at segmenting a stone while another type of machine learning may be better at segmenting a PAUC tool. In some implementations, results from one machine learning algorithm may be selected for the mask depending on the type of object suspected of being in the video. As described, supplemental data such as data collected by a robotic system can be used to narrow down the possible identifications for the object. In these situations, it may be possible to put more weight on results from machine algorithms that are better at identifying those types of object (e.g., by using a weighted average) or otherwise prioritizing the output from a particular machine algorithm in determining the final mask for the image.

### Student-Teacher Training

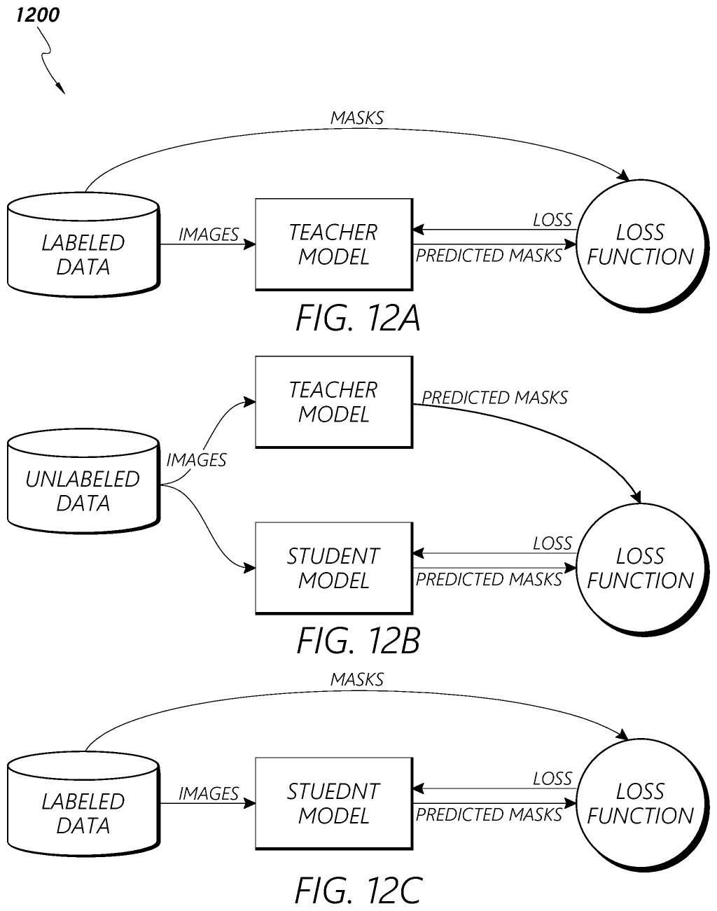

FIGS. 12A, 12B, and 12C illustrate student-teacher training paradigm 1200 in accordance with one or more embodiments. The object segmentation framework 1100 of FIG. 11 may be trained using the student-teacher training paradigm 1200. As a note, the student-teacher training paradigm 1200 described herein differentiates itself from student-teacher learning that involves training a smaller model to mimic the behavior or predictions of a larger, more complex model, often used to transfer knowledge gained from the complex model to the smaller model. Rather, the student-teacher training paradigm 1200 focuses on maximally leveraging unlabeled data and uses the same architecture for both its student and teacher.

The student-teacher training paradigm 1200, in addition to only using traditional supervised learning to train a model, further includes a self-supervised method to further improve the model. As a first step, a teacher model is trained using the traditional supervised learning with labeled data. The self-supervised method uses unlabeled data (e.g., data that have not yet been labeled), which may be synthetic data, to produce pseudo-labels using the trained teacher model. As a second step, a student model can be trained using the unlabeled data and the pseudo-labels. As a third step, after training the student model with the unlabeled data, the student model can be fine-tuned using the labeled data. The training paradigm 1200 is described in greater detail with FIGS. 12A, 12B, and 12C.

FIG. 12A illustrates the first step training the teacher model with labeled data. The teacher model is trained with the traditional supervised training methodology. Labeled data can include image data (e.g., the known actual image data 1111 of FIG. 11 and known masks (e.g., the known hard masks 1131 and/or the known soft masks 1132 of FIG. 11). In some implementations, the teacher model may be a randomly initialized model. A loss function of the teacher model can compare a predicted mask (e.g., a mask predicted by the teacher model for the image data) against the ground truth (e.g., a known mask corresponding to the image data) to improve accuracy of the teacher model.

FIG. 12B illustrates the second step training the student model with unlabeled data and the trained teacher model from FIG. 12A. The trained teacher model can create pseudo-labels from unlabeled data, where the pseudo-labels are predicted masks. In some embodiments, the student model may be of the same architecture as the teacher model but may not an exact copy of the teacher model. For instance, the student model may have the same number of neurons as the teacher model but the neurons may be initialized with different values. As shown, the student model can learn from the unlabeled data and the pseudo-labels by comparing its own predicted masks with the pseudo-labels. Accordingly, the student model can be trained without the labeled data during the second step.

FIG. 13C illustrates the third step further training the student model with labeled data. The student model can be exposed to the labeled data, which may be the same data involved in the first step, with supervised learning in the same manner as the first step. Accordingly, the student model can be fine-tuned with the actual image data and masks during the third step.

The student-teacher training paradigm 1200 can provide various advantages. Importantly, the student-teacher training paradigm 1200 leverages the power of unlabeled data which are more readily available than labeled data and allows for the student model to see more data than traditional supervised learning.

### Contextual Information: Metrics and Events

FIGS. 13A and 13B illustrate example metrics 1300 and events 1350 in accordance with one or more embodiments. In some embodiments, the example metrics 1300 and the events 1350 can be the metrics and events 790 in FIG. 7. Accordingly, the example metrics 1300 and the events 1350 can be a subset of contextual information generated by the contextual information generator module 834 of FIG. 8.

FIG. 13A illustrate the example metrics 1300 as categorized based on objects of interest in the first column, metric identifiers in the second column, and sources of data from which the example metrics 1300 are determined in the third column. Likewise, the example events 1350 are similarly categorized. With regard to the third column, “VISION” indicates that the particular associated metric is determined using endoscopic image data and “LOGS” indicates that the metric is determined using multi-modal data from the system logs. As described before, injecting additional information from endoscopic image data along with the multi-modal data coming from other parts of the robotic medical system can enable determination of additional metrics and events. Although two types of sources, “VISION” and “LOGS” are illustrated, it is contemplated that a metric or an event may be determined based on more or fewer sources and, in some instances, based on different combinations than those shown.

In FIG. 13A, example objects include a LASER, basket (e.g., forceps), PAUC, and stone. A LASER “number of activation” metric may be determined based on “VISION” by a neutral network that detects LASER frames (e.g., lasing frames). A LASER “tool usage time” metric may be determined based on “VISION” by aggregating lasing frames. In some implementations, the metric may be determined using a sliding window over a sequence of frames to account for dropped frames to estimate time the LASER was used. A LASER “protrusion length” metric may be determined based on “VISION” by determining a percent of space taken by LASER when the LASER is in a scene.

In another example, a basket “number of full passes” metric may be determined based on “VISION” and “LOGS.” One full pass may be defined as the activity of a basket entering the body, retrieving a stone, and exiting the body. A full pass can be counted by using the “VISION” depicting when a stone is being held along with the basket with each full pass increasing the count. “LOGS” can help distinguish whether the basket is entering or exiting with insertion and retraction commands. A basket “number of grasp attempts” may be determined based on “VISION” and “LOGS.” “LOGS” can supply when a grasp attempt is made open/close button inputs. “VISION” can be used to confirm the success of the attempt. A basket “moving with stone time” metric can be determined by examining “LOGS” for retraction/insertion joystick inputs for the basket and “VISION” to confirm stone movement with the basket. A basket “tool usage time” metric can be determined by aggregating the time taken from the above described basket metrics.

In another example, a PAUC “suctioning stone” metric may be determined by segmenting objects (e.g., a PAUC tip, a PAUC sheath, and a stone) within “VISION” and examining suctioning command from “LOGS.” A PAUC “active vs. passive time” metric may be determined based on “VISION” and “LOGS.” Passive time would be when the PAUC is in “VISION” with no commands from the logs while active time would be when there are, for example, pendant controller commands in “LOGS.” A PAUC “repositioning stone” metric can be determined by segmenting objects (e.g., a PAUC tip, a PAUC sheath, and a stone) within “VISION” with optical flow to track changes in visual states and articulation command from “LOGS.” A PAUC “tool usage time” metric may be determined by aggregating all of the active time taken from the above described PAUC metrics.

In yet another example, a stone “treatment time” metric may be determined by aggregating the time taken from each other took time taken metric. A stone “end of treatment” metric may be determined based on “VISION” by detecting when there are no stones seen for a period of time and providing a timestamp.

In FIG. 13B, example objects include a LASER, PAUC, needle, and UAS. For example, a LASER “misfires” event can be determined based on “VISION” by extracting LASER frames and further analyzing segmented objects based on shape information and image features.

In another example, a PAUC “blind driving” event can be determined based on “VISION” and “LOGS.” Detection of the PAUC body without the PAUC tip in “VISION” would indicate blind driving where the user is driving, as indicated by “LOGS,” without seeing the tip in view.

In another example, a needle “successful puncture” event may be determined based on “VISION” by detecting the needle during percutaneous access that indicates successful puncture. This can be further modified to include time windows from the “LOGS” for more accurate time and repeat punctures, or to detect unsuccessful punctures. A needle “backflow check” event can be determined by combining the “successful puncture” event based on “VISION” with EM data from “LOGS.” A backflow is where the fluid or medication can flow backward into the needle or syringe after the injection is complete and a backflow check may involve quickly retracting and reinserting the needle. The needle movement can be indicated by EM data from “LOGS.”

In yet another example, a UAS “fast retraction” event can be determined by detecting when UAS is in “VISION” frame and examining input commands from “LOGS.”

While the example metrics 1300 and events 1350 list various objects and their metrics and events, it is noted that other objects (e.g., anatomical features, treatment targets, medical tools, or the like) and related metrics and events are contemplated by the present disclosure.

### Multi-Modal Data Timeline

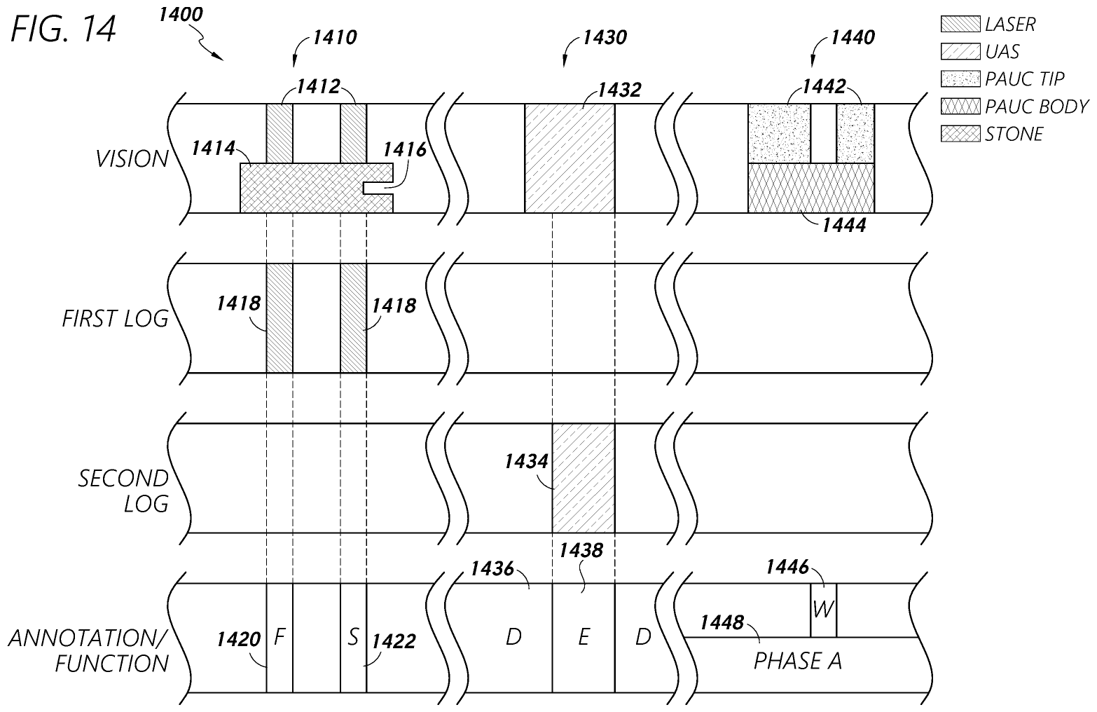

FIG. 14 illustrates an example timeline 1400 of multi-modal data and generated contextual information in accordance with one or more embodiments. Columns of the timeline 1400 show three events, a LASER “misfire” event 1410, a UAS “fast retraction” event 1430, and a PAUC “blind driving” event 1440. Each of the three event accesses multi-modal data (e.g., data from two or more sources of data). Rows of the timeline 1400 show three types of data (e.g., vision data, first log data, and second log data) and a determined annotation/function. In the timeline 1400, time flows from left to right. Legends on the top right of the timeline 1400 are used to indicate associations with certain referenced object of interest mapped by the legends.

The LASER “misfire” event 1410 accesses the vision data and the first log data. The accessed vision data depicts two LASER vision instances 1412 depicting a LASER and one stone vision instance 1414 depicting a stone. It is noted that the one stone vision instance 1414 is longer in time duration and wholly contains the two LASER vision instances 1412 in the timeline 1400. The accessed first log data shows two lasing command instances 1418 (e.g., a first lasing and a second lasing) that matches in time with the two LASER vision instances 1412. The one stone vision instance 1414 shows that (i) the stone does not change in size in response to the first lasing (e.g., a failed attempt 1420 denoted “F”) and that a stone portion 1416 is no longer depicted in response to the second lasing (e.g., a successful attempt 1422 denoted “S”). The results of both attempts can be annotated/stored in a data store, such as the data store 806 of FIG. 8.

The UAS “first retraction” event 1430 accesses the vision data and the second log data. The accessed vision data depicts one UAS vision instance 1432 depicting a UAS and the accessed second log data shows one UAS state instance 1434 representing a state of a distal tip of an endoscope positioned within the UAS. In the timeline 1400, it is noted that the one UAS state instance 1434 begins at some time after the one UAS vision instance 1432 first detects the UAS, thereby indicating that the distal tip transitions from outside the UAS to inside the UAS during the one UAS vision instance 1432. “First retraction” functionality can provide faster retraction when the distal tip is safely positioned within the UAS. Accordingly, the robotic medical system initialized with the “first retraction” functionality disabled 1436 (denoted “D”) may automatically enable 1438 (denoted “E”) the functionality when the distal tip is inside the UAS. When the UAS is no longer detected, the robotic medical system may automatically disable the functionality again. Such automatic functionality management may be performed by the functional manager module 850 of FIG. 8.

The PAUC “blind driving” event 1440 accesses the vision data and the annotation. The accessed vision data depicts two PAUC tip vision instances 1442 depicting a PAUC tip and one PAUC body instance 1444 depicting a PAUC body. In the timeline 1400, it is noted that the body vision instance 1444 wholly contains the tip vision instances 1442. The accessed annotation indicates that the PAUC is driven during “PHASE A” 1448 which here is assumed as a percutaneous phase. As described with the example events 1350 of FIG. 13B, “blind driving” can be determined based on detection of the PAUC body without the PAUC tip. Accordingly, a “blind driving” warning 1446 (denoted “W”) may be provided to a physician, for example on a display, for a time period the two PAUC tip vision instances 1442 does not match the ne PAUC body instance 1444. The annotation may have been generated and annotated by the metadata annotator module 836 of FIG. 8. In some implementations, the warning 1446 may be stored in the data store 806 as annotation.

Although only three events are shown, it is contemplated that any metrics and events, including the example metrics 1300 and events 1350, may be mapped in the timeline 1400 in a similar manner.

### Contextual Information Generation Flow

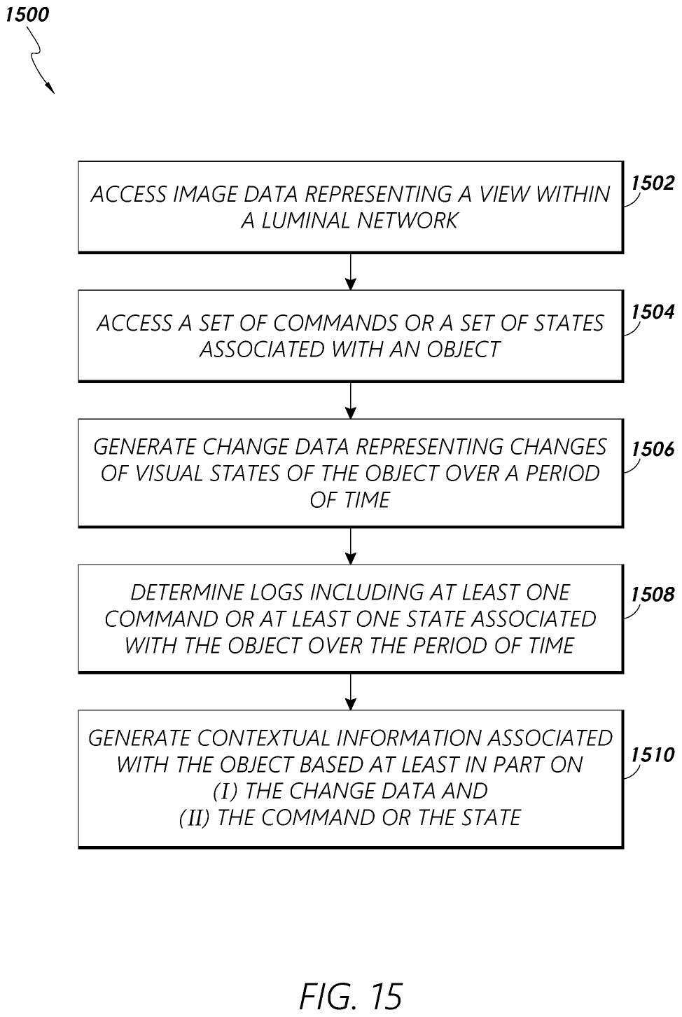

FIG. 15 illustrates a flow diagram illustrating a process 1500 for generating contextual information of an object in accordance with one or more embodiments. At block 1502, the process 1500 involves accessing image data representing a view within a luminal network. The image data can be one or more image frames (e.g., video frames) of vision data captured by a distally positioned camera of an endoscope. In some embodiments, the image data can be obtained in real-time from the camera or from an image repository storing past captured image data.

At block 1504, the process 1500 involves accessing a set of commands or a set of states associated with an object. In some embodiments, the object can be a medical tool or a portion thereof, an anatomical feature (e.g., a nodule), a target object (e.g., a kidney stone), or a background feature. The set of states can include kinematic states, visual states, phase/workflow states, result states, warnings/flag states, or the like. The set of commands or the set of states may be obtained in real-time from the robotic medical system or any portion thereof (e.g., sensors) or from a log repository.

At block 1506, the process 1500 involves generating change data representing changes of visual states of the object over a period of time. The visual states can be determined from one or more image frames of the image data. For example, LASER lasing (e.g., turning on) may be determined from an image frame where the change data may indicate the lasing. As another example, optical flow of the object may be determined from sequential images where the change data may indicate a motion of the object.

At block 1508, the process 1500 involves determining logs including at least one command or at least one state associated with the object over the period of time. The logs can provide sensor data, robot data, annotation data, or other data that can provide additional context when combined with the image data.

At block 1510, the process 1500 involves generating contextual information associated with the object based at least in part on (i) the change data and (ii) the command or the state associated with the object. In some embodiments, the contextual information may include the described metrics and events.

It is contemplated that the process 1500 may, in some instances, be executed online or in real-time, for example, to manage various functionalities of the robotic medical system as described in relation to the functional manager module 850 of FIG. 8. In other instances, the process 1500 may be executed offline to annotate existing image data in the image repository and to provide case indexing functionality of the insights module 840.

### ADDITIONAL EMBODIMENTS

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A system for extracting information of objects from video captured during a medical procedure, the system comprising:
an image repository configured to store image data representing views within a luminal network, the views captured with an imaging device;
a log repository configured to store commands and/or states associated with an object within the luminal network; and
control circuitry configured to:
generate change data representing changes of visual states of the object over a time period based at least in part on the image data;
access the log repository to determine logs including at least one command or at least one state associated with the object over the time period; and
generate contextual information associated with the object based at least in part on (i) the change data and (ii) the at least one command or the at least one state associated with the object.

2. The system of claim 1, wherein the control circuitry is further configured to:
assign, using a machine learning classifier, a semantic label to the object in one or more image frames of the image data.

3. The system of claim 1, wherein the control circuitry is further configured to:
determine the object is a LASER, a basket, a Percutaneous Antegrade Urethral Catheter (PAUC), a ureteral access sheath (UAS), a needle, an anatomical feature, or a stone.

4. The system of claim 1, wherein the control circuitry is further configured to:
determine a medical procedure or a phase of the medical procedure.

5. The system of claim 1, wherein the control circuitry is further configured to:
based on the change data, determine a starting image frame and an ending image frame from one or more image frames of the image data, the change data including at least one of: (i) visibility of the object, (ii) movement of the object, or (iii) a detected size, shape, or count of the object.

6. The system of claim 1, wherein the accessing the log repository further comprises:
filter the log repository to select the logs including the at least one command or the at least one state associated with the object;
determine a timestamp from the selected logs; and
determine a starting image frame for one or more image frames of the image data based on the timestamp.

7. The system of claim 1, wherein the control circuitry is further configured to:
select an image frame associated with a timestamp from one or more image frames of the image data; and
index the image frame with the determined contextual information, the contextual information including at least one of: (i) a medical procedure, (ii) a phase of the medical procedure, (iii) a result of the medical procedure, (iv) a result of the phase of the medical procedure, (v) a visual state of the image data, (vi) visibility of the object, or (vii) a relative position of the object in relation to another object.

8. The system of claim 7, the control circuitry is further configured to:
receive a selection query including the contextual information; and
in response to the receiving of the selection query, provide the timestamp or the image frame.

9. The system of claim 1, wherein the commands include at least one of: insertion, retraction, LASER activation, articulation, basket open or closure, aspiration, irrigation, or puncture.

10. The system of claim 1, wherein the states include at least one of: kinematics, position, orientation, usage time, number of activations, protrusion length, number of stone retrievals, treatment time, articulation duration, blind driving, backflow, LASER fires or LASER misfires, or successful puncture.

11. The system of claim 1, wherein the control circuitry is further configured to:
based on the determined contextual information, enable an operational functionality of the object.

12. The system of claim 1, wherein the control circuitry is further configured to:
cause a display to present a warning based on the determined contextual information.

13. The system of claim 1, wherein the log repository is configured to further store electromagnetic (EM) sensor data and the contextual information is generated based at least in part on the EM sensor data.

14. The system of claim 1, wherein the control circuitry is further configured to:
access a voice recording captured by a recording device;
converting the voice recording into text; and
indexing a first segment of the image data with a first segment of the text, the first segment associated with a timestamp.

15. The system of claim 14, the control circuitry is further configured to:
receive a selection query including the first segment of the text; and
in response to the receiving of the selection query, provide the timestamp or the first segment of the image data.

16. A method for extracting information of objects from image captured during a medical procedure, the method comprising:
accessing image data representing a view within a luminal network, the image data accessed from an image repository configured to store the image data;
accessing commands and/or states associated with a medical tool configured to operate within the luminal network from a log repository;
generating change data representing changes of visual states of an object over a time period based at least in part on the image data;
determining logs including at least one command or at least one state associated with the medical tool over the time period; and
generating contextual information associated with the object based at least in part on (i) the change data and (ii) the at least one command or the at least one state associated with the medical tool.

17. The method of claim 16, further comprising:
filtering the log repository to select the logs including the at least one command or the at least one state associated with the medical tool;
determining a timestamp from the selected logs; and
determining a starting image frame for one or more image frames of the image data based on the timestamp.

18. The method of claim 16, further comprising:
selecting an image frame associated with a timestamp from one or more image frames of the image data; and
indexing the image frame with the determined contextual information, the contextual information including at least one of: (i) the at least one command or (ii) the at least one state, the at least one command or the at least one state associated with the medical tool.

19. The method of claim 18, further comprising:
receiving a selection query including the contextual information; and
in response to the receiving of the selection query, providing the timestamp or the image frame.

20. A system for determining metrics and events of objects from image captured during a medical procedure, the system comprising:
control circuitry communicatively coupled to (i) an image repository configured to store image data representing views within a luminal network, the views captured with an imaging device, and (ii) a log repository configured to store data from sensors other than the imaging device, the control circuitry configured to:
generate change data representing changes of visual states of an object over a time period based at least in part on the image data;
access the log repository to determine logs including sensor data associated with the object over the time period; and
determine metrics and events associated with the object based at least in part on (i) the change data and (ii) the sensor data associated with the object.
